(19) United States
(12) Patent Application Publication
(10) Pub. No.: US 2016/0100850 A1
(43) Pub. Date: Apr. 14, 2016

(54) TONGUE CLEANER

(71) Applicant: Shikien Co., Ltd., Niigata (JP)

(72) Inventor: Michio Tanaka, Niigata (JP)

(73) Assignee: Shikien Co., Ltd., Niigata (JP)

(21) Appl. No.: 14/977,089

(22) Filed: Dec. 21, 2015

Related U.S. Application Data
(63) Continuation-in-part of application No. 13/253,653, filed on Oct. 5, 2011.

(30) Foreign Application Priority Data
Oct. 8, 2010 (JP) PCT/JP2010/067769
Feb. 22, 2011 (JP) 2011-035359

(51) Int. Cl. A61B 17/24

(52) U.S. Cl. CPC A61B 17/244

(57) ABSTRACT

Provided is a tongue cleaner comprising: a head 1 forming a main body of said tongue cleaner; and a plurality of linear materials 31, 41, 51, 61 protruding from said head 1 and comprised of a loop pile; wherein said plurality of linear materials make up a plurality of linear material groups 65 to 69 comprised of at least two of said linear materials 31, 41, 51, 61 protruding from a pair of common loop-starting points 71, and a loop shape of said linear material 31, 41, 51, 61 differs from one another in each linear material group 65 to 69.

### CROSS-REFERENCE TO RELATED APPLICATIONS

This application is a Continuation-in-Part of U.S. Non-Provisional application Ser. No. 13/253,653 filed on Oct. 5, 2011, the benefit of which is hereby claimed under 35 U.S.C. section 120, and is further incorporated herein by reference. This application also claims priority to Japanese patent application No. 2011-035359 filed on Feb. 22, 2011 and International application PCT/JP2010/067769 filed on Oct. 8, 2010, which are herein incorporated by reference in their entirety.

### TECHNICAL FIELD

The present invention relates to a tongue cleaner.

### BACKGROUND ART

Heretofore, as this sort of tongue cleaner, there has been proposed a tongue cleaner including a head and a stick-shaped handle connected to one end of the head. In the tongue cleaner, the head includes, on one of its front and reverse faces, a concavely curved surface with a valley portion extending parallel to the longitudinal direction of the stick-shaped handle. Further, the head is formed in a flat and thin shape, having a substantially uniform thickness, and the other face of the head is formed into a convexly curved surface. Then, a sheet composed of a twill-woven textile, a sateen-woven textile and a pile-woven textile is attached to both the front and reverse faces of the head. When cleaning tongue coating on both lingual sides using this tongue cleaner, the lingual raised sides are allowed to fit in the concavely curved surface, thus enabling tongue coating to be cleaned without shifting the tongue cleaner laterally, simply by moving the tongue cleaner back and forth with the stick-shaped handle held in hand and the concavely curved surface brought into contact with the tongue.

Further, as an improvement to the above-mentioned conventional art, there has been proposed the one in which said head is configured such that a sheet is provided on a surface of a core material formed with a concavely curved surface, the sheet being fixed on a lateral side of the core material; the concavely curved surface and the sheet opposed to the concavely curved surface are formed so as to be slightly slidable relative to each other; and when moving the tongue cleaner back and forth with its head in contact with a tongue, the grime behind tongue coating can be scraped out while raising tongue coating by the sheet that is slightly slidable relative to the head. At this time, the sheet opposed to each of the concavely curved surface and convexly curved surface becomes slidable, thus enabling the sheet to softly contact with the tongue, while permitting the tongue to be less affected by a possible strong force applied to the stick-shaped handle owing to this sliding motion of the sheet.

Further, heretofore, there has been disclosed another tongue cleaner in which a loop pile fabric whose pile surface is composed of multifilament yarns is fixed to a tip of a stick. Besides, various patent publications disclose tongue cleaners which employ looped piles.

Patent document 1: Japanese unexamined patent application publication No. 2008-188275.

Patent document 2: US unexamined patent application publication No. 2009-131960.

Patent document 3: Japanese unexamined utility model publication No. 2515465.

Patent document 4: US patent publication No. 5938673.

In the conventional art according to the above-mentioned patent document 2, the convexly curved surface and the sheet opposed to the convexly curved surface are always in close contact with each other so that the sheet is able to slide on the convexly curved surface. In contrast, the concavely curved surface and the sheet opposed to the concavely curved surface are formed with a gap therebetween while it is being in pristine condition, and the sheet is then slightly stretched as a user continues to use the tongue cleaner so that the sheet comes in close contact with the concavely curved surface to become slidable thereon.

As a result, a gap is formed between the concavely curved surface and the sheet opposed to the concavely curved surface in pristine condition, thus leading to the likelihood of causing tongue coating to accumulate in the gap.

For this reason, one may consider, for example, bending the sheet beforehand so as to permit the same to go along in parallel with the concavely curved surface so that no, or the slightest possible gap, if any, may be formed between the concavely curved surface and the sheet opposed to the concavely curved surface, which, however, would result in not only the increase of the manufacturing steps but the difficulties in forming the sheet so as to allow the same to go precisely along the concavely curved surface.

When removing the grime in tongue coating in such a way as were disclosed in the patent documents 3, 4, a loop is allowed to contact with a tongue and then move along a surface of the tongue, thus scraping out the grime hidden in the tongue coating.

Whereas, there exist a number of minute mucosal protrusions, called tongue papillae, on a rear side, sides, etc. of a tongue. These tongue papillae are, e.g., on the order of 0.5 to 1 mm in size.

According to the conventional tongue cleaners, when scraping out the grime attached to tongue papillae with the loop, there is a possibility of injuring the tongue papillae with the loop.

Therefore, a problem to be solved by the invention is to form, from the beginning, no or the slightest possible gap between the concavely curved surface formed in a head and the sheet covering the concavely curved surface, thereby making it less likely for tongue coating to accumulate in the gap. Further, another problem to be solved by the invention is to manufacture a tongue cleaner either without such gap or with the slightest possible one.

Further, with respect to a tongue cleaner provided with a plurality of filiform materials for the sake of scraping out tongue coating, the present invention provides a tongue cleaner enabling resultant scratches on a tongue to be as few as possible and the grime of the tongue coating to be surely rubbed off without failing to do so.

A first aspect of the present invention is a tongue cleaner comprising: a head forming a main body of said tongue cleaner; and a plurality of linear materials protruding from said head and comprised of a loop pile; wherein said plurality of the linear materials make up a plurality of linear material groups comprised of at least two of said linear materials protruding from a pair of common loop-starting points, and a loop shape of said linear material differs from one another in each linear material group.

A second aspect of the present invention is the tongue cleaner, wherein each linear material in the plurality of the linear material groups has a random height, random leaning directions toward both horizontal and front-back directions as well as a random orientation of the loop formed by said linear material.

A third aspect of the present invention is the tongue cleaner, wherein each linear material in the plurality of the linear materials has a random height, random leaning directions toward both horizontal and front-back directions as well as a random orientation of the loop formed by said linear material.

A fourth aspect of the present invention is the tongue cleaner, wherein the orientation of each loop is so random as to cover substantially all directions in each linear material group.

A fifth aspect of the present invention is the tongue cleaner, wherein the orientation of each loop is so random as to cover substantially all directions in the linear materials.

A sixth aspect of the present invention is the tongue cleaner, wherein the plurality of the linear material groups include first and second linear material groups, and at least one linear material of the first linear material group overlaps at least one linear material of the second linear material group with respect to a moving direction (Z₁) of said tongue cleaner.

A seventh aspect of the present invention is the tongue cleaner, wherein the plurality of the linear material groups include first and third linear material groups, and at least one linear material of the first linear material group overlaps at least one linear material of the third linear material group with respect to a direction parallel with a width defined as a distance between the pair of the common loop-starting points.

A eighth aspect of the present invention is the tongue cleaner, wherein the plurality of the linear material groups include at least one linear material group in which a width (W) defined as a distance between a leftmost end and a rightmost end in said one linear material group is larger than a width (w) defined as a distance between the pair of the common loop-starting-points therein.

According to the first aspect of the present invention, a gap can be minimized, and due to the smaller gap, the grime of the tongue coating enables to be scraped out more efficiently by using whole loop-pile, thereby enabling the grime of the tongue coating left without being scraped out to be reduced.

According to the second aspect of the present invention, the linear material is formed in a three-dimensional shape, thereby the grim of the tongue coating to be more reliably rubbed off.

According to the third aspect of the present invention, a gap can be minimized, and due to the smaller gap, the grime of the tongue coating enables to be scraped out more efficiently.

According to the fourth aspect of the present invention, the grime of the tongue coating can be surely rubbed off even if the tongue cleaner is moved to any directions.

According to the fifth aspect of the present invention, the grime of the tongue coating can be surely rubbed off even if the tongue cleaner is moved to any directions.

According to the sixth aspect of the present invention, a gap with respect to the moving direction Z₁ can be minimized, thereby enabling the grime of the tongue coating to be scraped out more efficiently, and thereby enabling the grime of the tongue coating left without being scraped out to be reduced.

According to the seventh aspect of the present invention, a gap with respect to the direction parallel with the width defined as the distance between the pair of the common loop-starting points can be minimized, thereby enabling the grime of the tongue coating to be coating to be scraped out more efficiently, and thereby enabling the grime of the tongue coating left without being scraped out to be reduced.

According to the eighth aspect of the present invention, the grime of the tongue coating in wider areas can be rubbed off at once.

### BRIEF DESCRIPTION OF DRAWINGS

FIG. 1 is a perspective view illustrating a tongue cleaner according to a first embodiment of the present invention.

FIG. 2 is a cross-sectional view on a line A-A in FIG. 1.

FIG. 3 is a cross-sectional view on a line B-B in FIG. 1.

FIG. 4 is a cross-sectional view illustrating a main section according to the first embodiment of the present invention.

FIG. 5 is a cross-sectional view illustrating the tongue cleaner in actual use according to the first embodiment of the present invention.

FIG. 6 is a cross-sectional view illustrating a first step of manufacturing a head of the first embodiment of the present invention.

FIG. 7 is a cross-sectional view illustrating a second step of manufacturing the above head.

FIG. 8 is a plan view illustrating a mold in which the above head is set.

FIG. 9 is a vertical cross-sectional view illustrating the mold in which the above head is set.

FIG. 10 is a cross-sectional view illustrating a first step of manufacturing a head according to a second embodiment of the present invention.

FIG. 11 is a cross-sectional view a second step of manufacturing the above head.

FIG. 12 is a vertical cross-sectional view illustrating a mold in which the above head is set.

FIG. 13 is a perspective view illustrating a tongue cleaner according to a third embodiment of the present invention.

FIG. 14 is an enlarged vertical cross-sectional view illustrating a main section according to a fourth embodiment of the present invention.

FIG. 15 is an enlarged plan view illustrating a usage condition according to the fourth embodiment of the present invention.

FIG. 16 is an enlarged vertical cross-sectional view illustrating the main section according to a fifth embodiment of the present invention.

FIG. 17 is an enlarged plan view illustrating a usage condition according to the fifth embodiment of the present invention.

FIG. 18 is an enlarged vertical cross-sectional view illustrating a main section according to a sixth embodiment of the present invention.

FIG. 19 is an enlarged perspective view illustrating a partially cut out section according to a seventh embodiment of the present invention.

FIG. 20 is an enlarged lateral view illustrating linear materials according to an eighth embodiment of the present invention.

FIG. 21 is an enlarged perspective view illustrating a plurality of linear material groups according to the eighth embodiment of the present invention.

FIG. 22 is an enlarged plan view illustrating a linear material group according to the eighth embodiment of the present invention.

FIG. 23 is a transverse plan view illustrating the linear material group according to the eighth embodiment of the present invention.

FIG. 24 is an enlarged elevational view illustrating the linear materials according to the eighth embodiment of the present invention.

FIG. 25 is an enlarged perspective view illustrating an orientation of a loop formed by the linear materials according to the eighth embodiment of the present invention.

FIG. 26 is an explanation drawing illustrating an orientation of a loop formed by the linear material according to the eighth embodiment of the present invention.

FIG. 27 is an explanation drawing illustrating the orientation of the loop formed by the linear material according to the eighth embodiment of the present invention.

FIG. 28 is an explanation drawing illustrating the orientation of the loop formed by the linear material according to the eighth embodiment of the present invention.

FIG. 29 is a perspective view illustrating a rotating brush according to the eighth embodiment of the present invention.

FIG. 30 is a perspective view illustrating the rotating brush being pressed against a sheet according to the eighth embodiment of the present invention.

FIG. 31 is a lateral view illustrating the rotating brush being pressed against the sheet according to the eighth embodiment of the present invention.

FIG. 32 is an enlarged vertical cross-sectional view illustrating a main section according to the eighth embodiment of the present invention.

FIG. 33 is a first figure illustrating how a grime of a tongue coating is rubbed off by the tongue cleaner of the conventional art.

FIG. 34 is a second figure illustrating how the grime of the tongue coating is rubbed off by the tongue cleaner of the conventional art.

FIG. 35 is a third figure illustrating how the grime of the tongue coating is rubbed off by the tongue cleaner of the conventional art.

FIG. 36 is a first figure illustrating how a grime of a tongue coating is rubbed off by the tongue cleaner according to the present invention.

FIG. 37 is a second figure illustrating how the grime of the tongue coating is rubbed off by the tongue cleaner according to the present invention.

FIG. 38 is a third figure illustrating how the grime of the tongue coating is rubbed off by the tongue cleaner according to the present invention.

FIG. 39 is a first figure illustrating how a grime of a tongue coating is rubbed off by the tongue cleaner of the conventional art.

FIG. 40 is a second figure illustrating how the grime of the tongue coating is rubbed off by the tongue cleaner of the conventional art.

FIG. 41 is a third figure illustrating how the grime of the tongue coating is rubbed off by the tongue cleaner of the conventional art.

FIG. 42 is a first figure illustrating how a grime of a tongue coating is rubbed off by the tongue cleaner according to the present invention.

FIG. 43 is a second figure illustrating how the grime of the tongue coating is rubbed off by the tongue cleaner according to the present invention.

FIG. 44 is a third figure illustrating how the grime of the tongue coating is rubbed off by the tongue cleaner according to the present invention.

Preferred embodiments of the present invention are described with reference to the accompanying drawings. It should be noted that the embodiments described below are not to limit the scope of the present invention set forth in the claims. Further, all the schemes described below are not necessarily the requirements of the present invention.

### FIRST EMBODIMENT

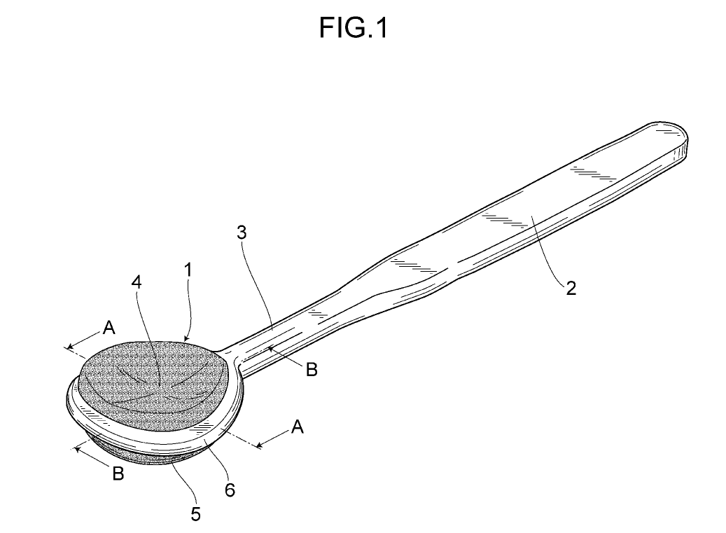

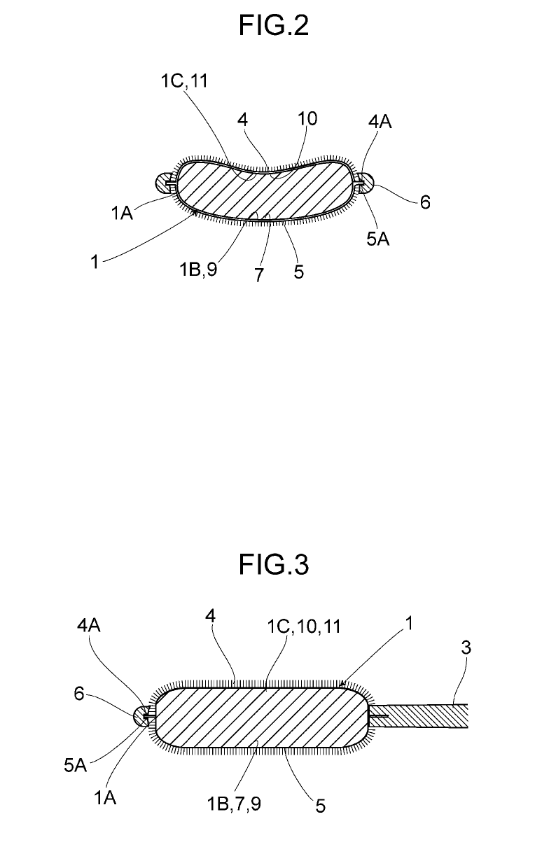

As shown in FIG. 1 to FIG. 5, a tongue cleaner includes an substantially straight stick-shaped handle 2 in a head 1 forming a main body of a cleaner. The head 1 and the stick-shaped handle 2 are connected with each other via a thin and slightly flexible neck 3. The head 1 is formed in a flat-and-thin shape and a face of the head 1 is covered with first and second sheets 4, 5 each having a surface roughened. The head 1 includes a ring 6 which is fixed to an outside of a lateral side 1A of the head 1 via the sheets 4, 5 and is connected to an end of a neck 3. The head 1 has a flattened and substantially isosceles triangle shape or equilateral triangle shape in plan view such that it is widened at its distal end and narrowed at its neck 3 side. In the present embodiment, the head 1 is formed in a substantially equilateral triangle shape whose corners are rounded. The head 1 has a first surface on a flat portion 1B side, e.g., an underside in FIG. 2, formed into such a convexly curved surface 9 that a raised portion 7 extends along a longitudinal direction of the stick-shaped handle 2 so as to fit the surface of a tongue 8. On the other hand, the head 1 has a second surface on a flat portion 1C side, e.g., an upper side in FIG. 2, formed into such a concavely curved surface 11 that a valley portion 10 extends along the longitudinal direction of the stick-shaped handle 2 so as to fit the surface of the tongue 8.

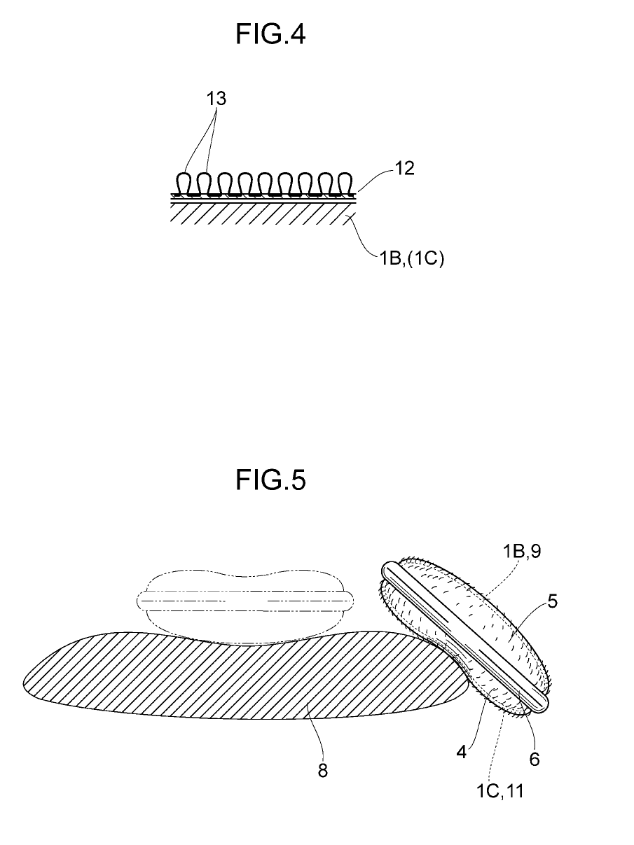

Then, the above concavely curved sheet 4 having a concavely curved vertical cross section is provided such that it is attached substantially in close contact with the concavely curved surface 11 or with a slight gap between the sheet 4 and the concavely curved surface 11. Further, the convexly curved sheet 5 having a convexly curved vertical cross section is provided such that it is attached in close contact with the convexly curved surface 9. As shown in FIG. 4, the sheets 4, 5 are provided with fibers 13 on the surface of a basal main body 12 thereof in a manner protruding outward toward the top and bottom, respectively. According to the present embodiment, a pile-woven textile is used such that the sheet 5 is provided so as to cover one flat portion 1B on the head 1 and a first half vertical portion on the lateral side 1A, while the sheet 4 is provided so as to cover the other flat portion 1C on the head 1 and a second half vertical portion on the lateral side 1A. Then, the edge 4A of the sheet 4 and the edge 5A of the sheet 5 are mated with each other by fitting the ring 6 on the lateral side 1A at the distal, lateral and neck 3 sides of the head 1 to thereby integrate the head 1, the edge 4A of the sheet 4, the edge 5A of the sheet 5 and the ring 6 with one another. It should be noted that the sheets 4, 5 may be formed from woven sheets including twill-woven textile, sateen-woven textile, etc., unwoven textile, or a surface-roughened material such as a porous material or a multi-void material, other than from a pile-woven textile.

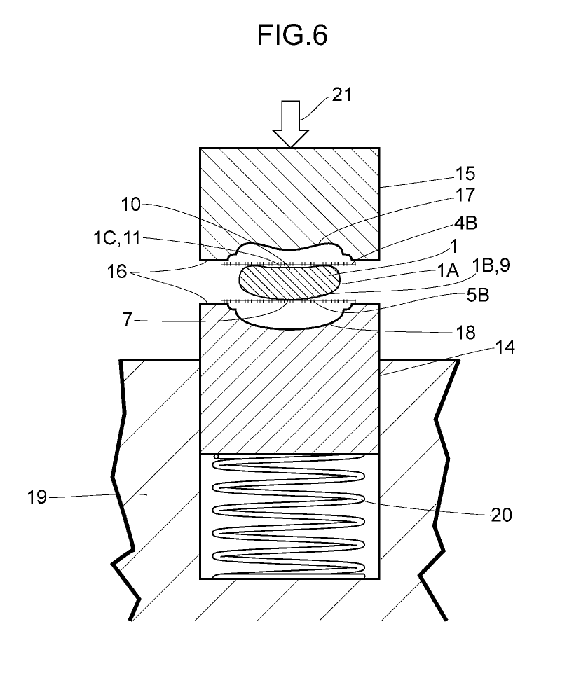

Next is a description of a method for manufacturing the tongue cleaner. In manufacturing the head 1, as shown in FIG. 6, there are provided a pair of a lower mold 14 and an upper mold 15 opposed to the lower mold 14 so as to be unitable with and separable from the lower mold 14. A concavely curved surface molding concave portion 17 with the same shape as that of the concavely curved surface 11 is formed on the underside of the upper mold 15, being one of the pair of divided surfaces 16, while a convexly curved surface molding concave portion 18 with the same shape as that of the convexly curved surface 9 is formed on the upper side of the lower mold 14, being the other of the pair of divided surfaces 16. Here, the lower mold 14 is supported on a pedestal 19 via an elastic means 20 such as an upwardly-urging spring or the like, while the upper mold 15 is provided with a pressing means 21 such as a press mechanism, etc.

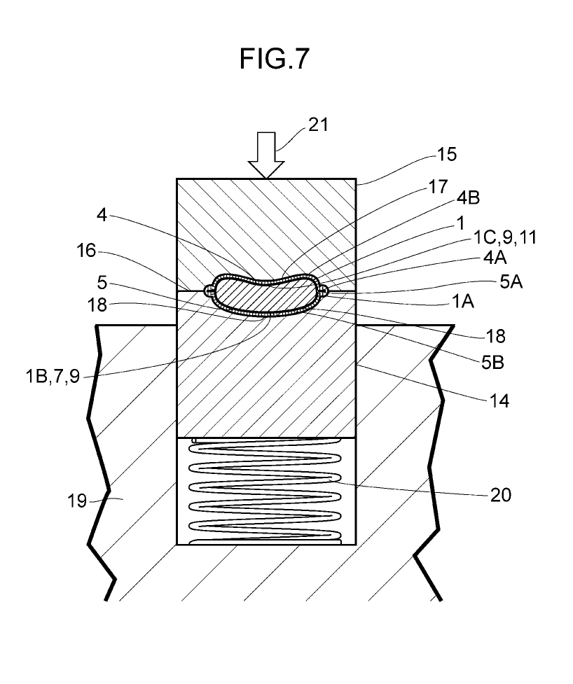

Then, a sheet material 5B made of a flat pile-woven textile for forming the sheet 5 and having its fibers 13 directed downwards, the head 1 having the convexly curved surface 9 directed downwards, and a sheet material 4B made of a flat pile-woven textile for forming the concavely curved sheet 4 and having its fibers directed upward are sequentially stacked, and thereafter the pressing means 21 is activated to press the upper mold 15 to the lower molds 14, whereby the sheet material 5B, the head 1 and the sheet material 4B are, as shown in FIG. 7, sandwiched, with pressure load being applied thereto, between the convexly curved surface molding concave portion 18 and the concavely curved surface molding concave portion 17. As a result, the sheet material 4B is sandwiched between the concavely curved surface molding concave portion 17 and the concavely curved surface 11 to be formed into a curved shape so as to form the valley portion extending in the longitudinal direction along the concavely curved surface 11 to thereby form the concavely curved sheet 4. At the same time, the sheet material 5B is sandwiched between the convexly curved surface molding concave portion 18 and the convexly curved surface 9 to be formed into a curved shape so as to form the raised portion extending in the longitudinal direction along the convexly curved surface 9 to thereby form the convexly curved sheet 5. Further, the respective edges 4A, 5A of the sheets 4, 5 are allowed to be coupled to each other on the lateral side 1A of the head 1 and the outside portions of the coupled edges are cut off.

Then, the mold is opened to separate the divided surfaces 16 from each other and then the integrated piece comprising the head 1, the concavely curved sheet 4 and the concavely curved sheet 5 is taken out. In this head 1, the sheet 4 molded into a concavely curved shape is either in close contact with the concavely curved surface 11 or with a slight gap formed therebetween and therefore the sheet 4 is not fixed to the concavely curved surface 11 so as to be slightly slidable on the concavely curved surface 11. Likewise, the sheet 5 molded into a convexly curved surface is either in close contact with the concavely curved surface 11 or with a slight gap formed therebetween and therefore the sheet 5 is not fixed to the convexly curved surface 9 so as to be slightly slidable on the convexly curved surface 9.

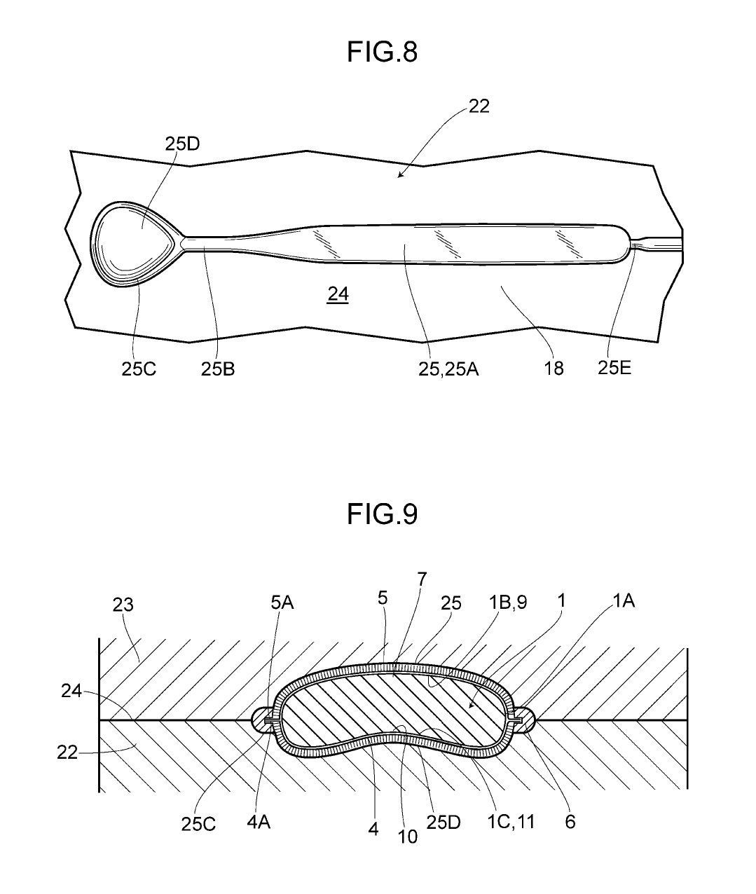

Next, the stick-shaped handle 2 and the ring 6 are molded using an injection mold shown in FIG. 8 and FIG. 9. The molding performed using this injection mold is to mold the stick-shaped handle 2, the neck 3, and the ring 6 integrally with the head 1 that has been integrated with the sheets 4, 5, with such head 1 being nested thereinside. Accordingly, the injection mold includes a lower mold 22 and an upper mold 23 which are unitable with and separable from each other, and a cavity 25, acting as a molding space formed in a halved manner on the respective divided surfaces 24 of the upper and lower molds 24, 25, comprises a ring molding section 25C formed at a frontal portion of a stick-shaped handle molding section 25A via a neck molding section 25B. Further, a placing section 25D for the preformed head 1 integrated with the sheets 4, 5 is formed inside this ring molding section 25C. This placing section 25D is formed in the same shape as the concavely curved surface 11 so that the sheet 4, which has already been integrated with the head 1 and formed in a concavely curved shape, can engage therewith. In addition, the cavity 25 of the upper mold 15 opposed to the placing portion 25D is formed in the same shape as the convexly curved surface 9. Furthermore, a feed opening 25E for feeding molten resin is formed at an end of the stick-shaped handle molding section 25A.

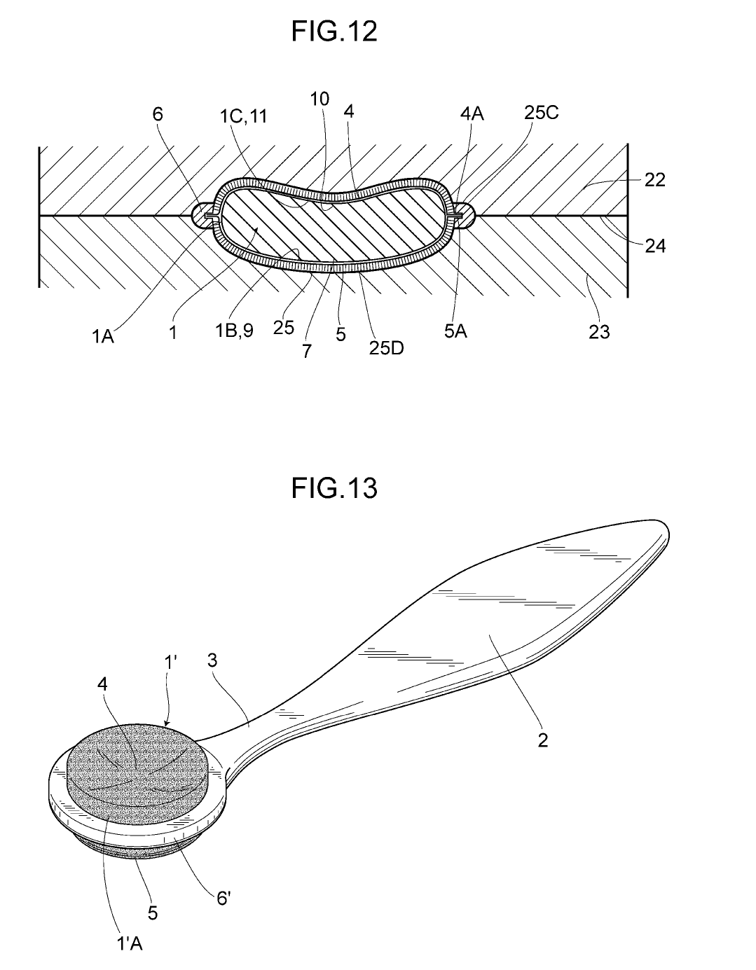

Accordingly, with the injection mold being opened, the head 1 is placed on the placing section 25D on an upper surface of the lower mold 22 with the sheet 4 placed at the underside and the sheet 5 placed at the upper side and then the injection mold is closed. Then, the molten resin fed from the feed opening 25E is allowed to fill the stick-shaped handle molding section 25A, the neck molding section 25B and the ring molding section 25C. At this moment, the resin filled in the ring molding section 25C comes in contact with the lateral side 1A of the head 1 and thus the ring 6 is allowed to be fitted to an outside of the lateral side 1A of the head via the edges 4A, 5A of the sheets 4, 5, thus permitting the head 1 to be fixed to an inside of the ring 6. Alternatively, as shown in FIG. 12 referred to hereinafter, the head 1 may be placed on the placing portion 25D with the sheet 4 placed at the upper side and the sheet 5 placed at the downside.

Then, after subjecting the molding to cooling, the mold is opened to take out a product. It should be noted herein that in the product thus manufactured, the concavely curved sheet 4 molded in a concavely curved shape is either in close contact with the concavely curved surface 11 or with a slight gap formed therebetween, while the sheet 4 is not fixed to the concavely curved surface 11 so that it is made slightly slidable on the concavely curved surface 11. Similarly, the convexly curved sheet 5 molded in a convexly curved shape is either in close contact with the convexly curved sheet 5 or with a slight gap formed therebetween, whilst the convexly curved sheet 5 is not fixed to the convexly curved surface 9 so that it is made slightly slidable on the convexly curved surface 9.

Consequently, as shown in FIG. 5, when grasping the stick-shaped handle 2 to let the head 1 enter a mouth and apply the head 1 to a convex portion of a tongue 8 such as a corner thereof, the head 1 is allowed to contact with the tongue 8 with the sheet 4, attached to the concavely curved surface 11, being faced downwards, thus scraping out tongue coating. In this case, the sheet 4 provided outside relative to the concavely curved surface 11 of the head 1 is slightly slidable without a gap and hence even if somewhat a large force is applied to the stick-shaped handle 2, such force is not transmitted at once from the head 1 to the sheet 4 but it is relaxed due to the sliding motion of the sheet 4, thus enabling the likelihood of injuring the tongue 8 to be reduced. Further, the sheet 4 is slidably attached in close contact with the concavely curved surface 11 in advance in a manner extending along the concavely curved surface 11 and hence there is no gap between the sheet 4 and the concavely curved surface 11, enabling the likelihood of the accumulation of tongue coating to be reduced.

Further, as shown with chain double-dashed lines in FIG. 5, the sheet 5 on the convexly curved surface 9 is applied to a place where the concavity of the tongue 8 exists. In that case, the convexly curved sheet 5 provided on the outside of the convexly curved surface 9 of the head 1 is able to be slightly slidable thereon, and therefore even if somewhat large force is applied to the stick-shaped handle 2, such force is not transmitted at once from the head 1 to the sheet 5 such that it is buffered due to the sliding motion of the sheet 5, thus permitting the likelihood of injuring the tongue 8 to be reduced.

Then, after use, the head 1 is washed with water and then it is dried.

As described above, according to the present embodiment, the first sheet 4 is provided on the face of the head 1 formed with the concavely curved surface 11 and besides it is fixed to the lateral side 1A of the head 1 to thereby enable the sheet 4 opposed to the concavely curved surface 11 to slide. Furthermore, the second sheet 5 is provided on the face of the head 1 formed with the convexly curved surface 9 and besides it is fixed to the lateral side 1A of the head 1 to thereby enable the sheet 5 opposed to the convexly curved surface 9 to slide. Therefore, the sheets 4, 5 are allowed to come in soft contact with the tongue 8, and even if a strong force is applied to the stick-shaped handle 2, it can be buffered by the sliding motions of the sheets 4, 5. Furthermore, since the sheet 4 is formed so as to be attached in close contact with the concavely curved surface 11 and thus the sheet 4 and the concavely curved surface 11 are allowed to be attached either in close contact with each other or with a slight gap therebetween without forming a large gap, the tongue coating scraped out becomes less likely to accumulate in the gap therebetween, thus permitting a good sanitary condition to be maintained.

Moreover, according to the foregoing method for manufacturing the tongue cleaner, the sheet material 4B of the sheet 4 arranged on the concavely curved surface 11 is pressed against the concavely curved surface 11 and thus the sheet material 4B is allowed to be in close contact with the concavely curved surface 11 or with a slight gap formed therebetween. Hence, the sheet 4 can be precisely formed. At the same time, the sheet material 5B of the sheet 5 arranged on the convexly curved surface 9 is pressed against the convexly curved surface 9 by the upper and lower molds 14, 15 and thus the sheet 5 is allowed to be in close contact with the convexly curved surface 9 or with a slight gap formed therebetween. Hence, in similar fashion to the sheet 4, the sheet 5 can be precisely formed, as well.

Yet more, after arranging the sheet material 4B on the concavely curved surface 11 with the head 1 being in a single-piece state as a component of the tongue cleaner, the sheet material 4B of the sheet 4 is pressed against the concavely curved surface 11 to form the sheet 4 into the curved surface in close contact with the concavely curved surface 11. Then, the ring 6 integrated with the stick-shaped handle 2 is provided on the lateral side 1A of the head 1 to fix the sheet 4 on the lateral side 1A of the head 1. As a result, the sheet 4 can be fitted so as to be precisely attached in close contact with the concavely curved surface 11 of the head 1 being in such a single-piece state or with a slight gap formed therebetween. Further, after arranging the sheet material 5B of the sheet 5 on the convexly curved surface 9 with the head 1 being in a single-piece state, the sheet material 5B of the sheet 5 is pressed against the convexly curved surface 9 by means of the upper and lower molds 14, 15 and thus the sheet 5 can be formed so as to be precisely attached in close contact with the convexly curved surface 9 of the head 1 being in a single-piece state or with a slight gap formed therebetween.

### SECOND EMBODIMENT

Hereunder is a description of other embodiments, in which the same numerical symbols are attached to the same parts as those of the first embodiment and their detailed descriptions are omitted.

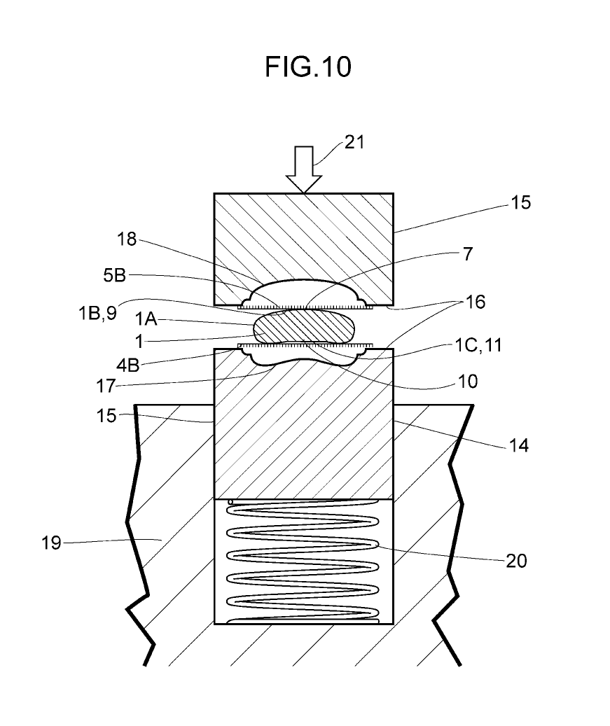

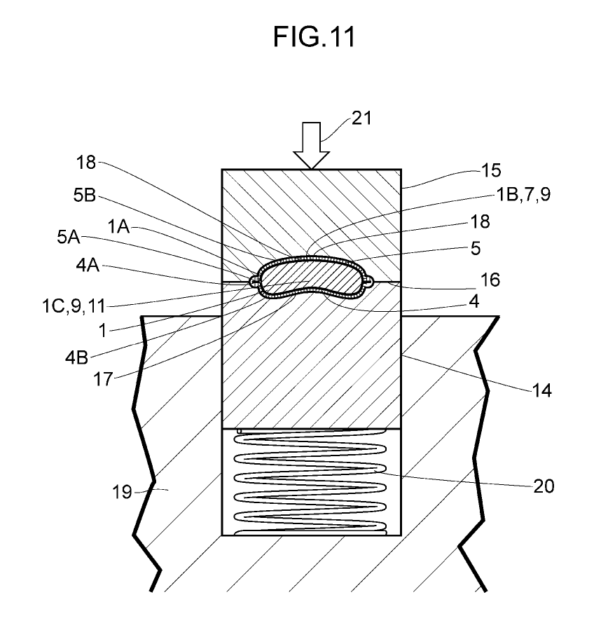

In a second embodiment shown in FIG. 10 to FIG. 12, unlike the first embodiment, the concavely curved surface molding concave portion 17 is formed on the lower mold 14 and the convexly curved surface molding concave portion 18 is formed on the upper mold 15. Further, as for the injection mold, the placing section 25D of the lower mold 22 is formed in the same shape as the convexly curved surface 9, and the placing section 25D of the upper mold 23 is formed in the same shape as the concavely curved surface 11.

Then, the sheet material 4B made of a flat pile-woven textile for forming the concavely curved sheet 4 and having its fibers 13 faced downwards, the head 1 with its convexly curved surface 9 faced upwards, and the sheet material 5B with its fibers 13 faced upwards are sequentially stacked. Thereafter, the pressing means 21 is activated to press the upper and lower molds 15, 14 and as a result, the integrated head is produced in the same manner as in the first embodiment with the sheets 4, 5 allowed to be in close contact with the head 1 or with a slight gap formed between the sheets 4, 5 and the head 1.

Further, the head 1 with the sheet 5 faced downwards and the sheet 4 faced upwards is placed on the placing section 25D and then the injection mold is closed to mold the stick-shaped handle 2, the neck 3 and the ring 6. In addition, as shown in FIG. 9, the concavely curved surface 11 may be placed on the placing section 25D to perform molding.

### THIRD EMBODIMENT

FIG. 13 shows a third embodiment, in which the same numerical symbols are attached to the same parts as those of the first embodiment and their detailed descriptions are omitted.

In the third embodiment, a plane of the head 1′ is formed in a circular shape. The ring 6′ is arranged on a lateral side l′A of this circular head 1′. In the third embodiment as well, the same operational effect as that obtained in the first embodiment can be attained. same operational effect as that obtained in the first embodiment can be attained.

### FOURTH EMBODIMENT

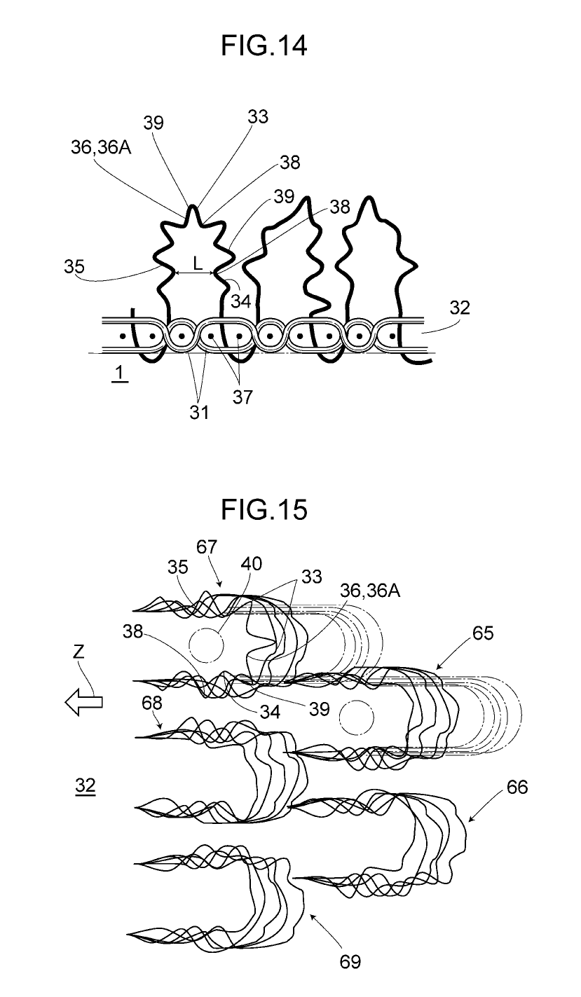

As shown in FIG. 14 and FIG. 15, in the fourth embodiment, loop-pile-woven textile is used which is made of multifilament yarns formed by overlapping a plurality of filaments. The multifilament yarns are constructed of plastics material such as nylon and so on. FIG. 14 is a cross-sectional view of the pile-woven textile. In the pile-woven textile, it is woven such that in part (a pile yarn 31) of warps (pile yarns 31, a base warp), a ring-like loop 33 is woven in a protruding fashion as a linear material on the surface of the pile-woven base textile 32 and therefore the pile yarn loop 33 is allowed to catch tongue coating to make the tongue coating easy to be raised, making it possible to more effectively scrape out the grime of the tongue coating. The innumerable loops 33 made of pile yarns, protruding in a ring-like manner on a surface of this pile-woven base textile 32 are attached to the head 1 in a direction intersecting with (desirably perpendicular to) a plane surface of the head 1. Then, the head 1 pressed against a tongue is allowed to move back and forth in the longitudinal direction of the head 1, defined as a moving direction Z, and thus the innumerable loops 33 made of the pile yarns, being warps rising from the surface of the pile-woven base textile 12 get entangled with the tongue coating to raise the tongue coating, making it easy to scrape out the grime. As such, the loop 33 made of the pile yarn includes a right and left pair of or a front and rear pair of protrusions 34, 35 which stand at an interval L and rise from the base textile 32; and a bridging portion 36 which bridges distal ends of the pair of the upright protrusions 34, 35. Note that numeral symbol 37 denotes a weft. In addition, the direction of the interval L between the protrusions 34, 35 intersects with the moving direction Z, which make a right angle in the present embodiment.

Further, the loop 33 includes a repeated pattern of a depressed portion 38 and a protruded portion 39 along the longitudinal direction of the loop 33 to form the longitudinal portion of the loop 33 into a corrugated shape. Alternatively, these depressed and protruded portions may be formed not along the entire length but only partially therealong.

Accordingly, when the head 1 is allowed to enter a mouth with the stick-like handle held by hand to apply the head 1 to the depressed portion of a tongue, the loop 33 is allowed to contact with the tongue, and thus the loop 33 falls down along the surface of the tongue to scrape out the grime of tongue coating. On the other hand, when the head 1 is allowed to enter the mouth with the stick-like handle held by hand to apply the head 1 to the protruded position of the corner or the like of the tongue, the head is allowed to contact with the tongue with the concavely-curved surface faced downwards to rub off the grime of the tongue coating in such a way as to scrape out the grime by the loop 33 made of the pile yarn 31.

When the loop 33 is moved in the moving direction Z to scrape out the grime of the tongue coating by the loop 33 in this way, tongue papillae 40, for example, may thrust into the loop 33 and the bridging portion 36 of the loop 33 may be caught by the tongue papillae 40. In that case, as shown by the dashed-dotted lines in FIG. 15, the corrugated portions of the depressed portion 38 and the protruded portion 39 in the loop 33 are allowed to extend linearly to act as a cushion and then the bridging portion 36 of the loop 33 is allowed to slide relative to the tongue papillae 40, thus permitting the bridging portion to slip through the tongue papillae 40 during this sliding motion. This slipping-through motion can reduce the possibility of injuring the tongue papillae 40 by the loop 33.

As for a manufacturing method for forming the depressed portion 38 and the protruded portion 39 along the longitudinal direction of the loop 33, it is performed in the following manner. A rotating brush, for example, is pressed against the looped textile 13 in an unprocessed state prior to being mounted on the head 1 as shown in FIG. 4 and then it is subjected to plastic processing to form depressed and protruded portions on the textile 13. Specifically, the pressure from the rotating brush or the like is applied to the textile 13 to deform the textile 13 over the limit of elasticity so that even after the pressure is removed, the deformation is left unchanged. The one processed thus way is attached to the head 1.

As described above, according to the fourth embodiment, there is provided the tongue cleaner in which a plurality of the loops is arranged on the head 1 as a linear material. The loop 33 is provided with the bridging portion 36 on the distal ends of the first and second protrusions 34, 35 located at intervals. The depressed portions 38 and the protruded portions 39 are provided on the bridging portion 36 along the longitudinal direction of the loop 33 to form the longitudinal direction of the loop 33 into a corrugated shape. Accordingly, when the loop 33 is caught by the tongue papillae 40, the loop 33 is pulled to linearly extend the depressed and protruded portions 38, 39 and thus the loop slips through the tongue papillae 40 without being forced to be thrust into the tongue papillae 40, thus permitting the possibility of injuring the tongue papillae 40 to be reduced.

Further, the approximately central portion 36A in the longitudinal direction of the bridging portion 36 is formed into a circular-arc shape protruding upward. Hence, if the tongue papillae 40 are located at the end of the bridging portion 36, the tongue papillae 40 can move relatively toward the approximately central portion 36A of the bridging portion 36 during the movement of the loop 33 toward to the tongue, and therefore the length over which the loop 33 can slip through the tongue papillae 40 can be enlarged, so that the loop 33 can slip through the tongue papillae 40 without injuring the tongue papillae 40.

Furthermore, all of the first and second protrusions 34, 35 and the bridging portions 36 are provided with the depressed portions 38 and the protruded portions 39 along the longitudinal direction of the loops 33 to form the longitudinal portions of the loops 33 into a corrugated shape. Hence, the extendable rate at which the depressed portions 38 and the protruded portions 39 get linear to extend the loops 33 is increased as much as possible and as a result the length over which the loop 33 can slip through the tongue papillae 40 can be more enlarged, so that the loop 33 can slip through the tongue papillae 40 without injuring the same.

Besides, by using the loop pile as the loop 33, the loop 33 is allowed to be pressed against the surface of a tongue with an appropriate elastic force, so that the tongue cleaner can be used without injuring the surface of the tongue even more.

Moreover, according to the foregoing manufacturing method, as a method for forming the depressed portions 38 and the protruded portions 39, there is proposed the pressing of the rotating brush, for example, against the looped textile to apply a plastic processing so as to form depressed and protruded portions on the looped textile, so that a simplified manufacturing method can be realized.

### FIFTH EMBODIMENT

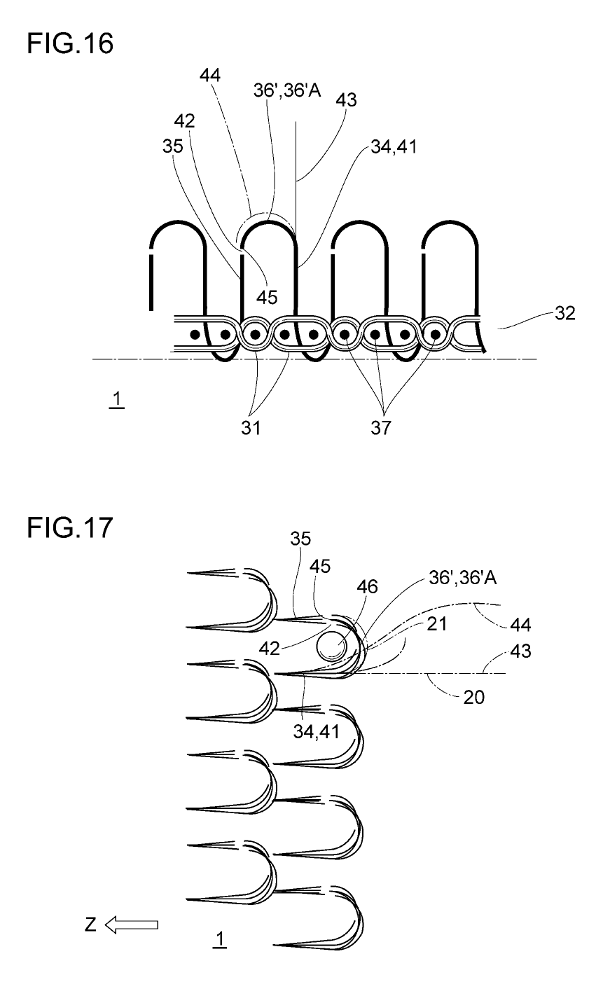

FIG. 16 and FIG. 17 show a fifth embodiment and specifically show a case where an improved pile-woven textile is used. A linear material 41 is provided so as to eliminate the continuity of any one of the first and second protrusions 34, 35 among these first and second protrusions 34, 35 and the bridging portion 36′ forming the rising portion arranged between the distal ends thereof. According to the present embodiment, a disconnected portion 42 is formed on one side of the distal end of the second protrusion 35 and thus the first protrusion 34 and the bridging portion 36′ are allowed to continuously extend in an inverted-J shape, whereas the second protrusion 35 is linearly formed. In addition, by forming the disconnected portion 42, the first and second protrusions 34, 35 are likely to get entangled with each other and therefore to prevent the occurrence of such tangle, a certain means (not shown) for preventing the linear material from coming away, such as bonding, welding or the like is desirably applied to the base textile 32.

As for an operation for forming the disconnected portion 42 in the loop 33, it is performed in the following manner. A cutter, e.g., is pressed against the looped textile 31 in a state of textile prior to being mounted on the head 1 as shown in FIG. 4 to thereby form the disconnected portion 42 in the textile 13. The one processed thus way is mounted on the head 1 as described above.

Accordingly, on a side of the distal end of the protrusion 34, a plurality of the protruding linear materials 41 provided on the main body of the cleaner are provided with the bridging portion 36′, being the rising portion, formed so as to intersect with a first longitudinal direction 43 of the protrusion 34 to allow a second longitudinal direction 44 to face sideways, while a distal end 45 of the rising portion 36′ is formed into a free end. An approximately central portion 36′A in the longitudinal direction 44 of the rising portion 36′ is formed in a circular-arc shape protruding upward, while the distal end 45 of the rising portion 36′ is folded toward the head 1 so that it is, as a whole, formed into the inverted-J shape. At this time, the width direction between the side of the distal end 45 and the protrusion 34 is defined so as to make a right angle with the moving direction Z.

As for a manufacturing method thereof, a cutting means such as a knife or the like (not shown) is applied to an upper end of the second protrusion 35 in the looped textile to form the disconnected portion 45.

Accordingly, when the head is allowed to enter a mouth with the stick-like handle held by hand to come in contact with a tongue and then the head 1 is moved along the moving direction Z, the linear material 41 is allowed to fall along the surface of a tongue, thus rubbing off the grime of tongue coating so as to scrape out the grime by the sideways rising portion 36′ of the linear material 41.

When the grime of tongue coating is scraped out by moving the linear material 41 in the moving direction Z, and then when tongue papillae 46 thrust into the rising portion 36′, e.g., and the rising portion 36′ is caught by the tongue papillae 46, as shown in a dashed-dotted lines of FIG. 17, then, the angle between the first longitudinal direction 42 and the second longitudinal direction 43 increases so that the cantilevered rising portion is paralleled with the first longitudinal direction 42 by the tongue papillae 46 and as a result, the distal end 45 acting as a free end is allowed to slip through the tongue papillae 46, preventing the tongue papillae 46 from being injured.

As described above, according to the foregoing embodiment, on the side of the distal end of the protrusion 34, the linear material 41 is provided with the longitudinal part of the rising portion 36′ in a direction intersecting with the longitudinal direction 42 of the protrusion 34 and the distal end 45 of the rising portion 36′ is formed into a free end. Hence, when the rising portion 36′ is caught by the tongue papillae 46, the rising portion 36′ is pulled by the tongue papillae 46 to deform along the first longitudinal direction 42 of the protrusion 41 and thus the rising portion 36′ can slip through the tongue papillae 46, so that the possibility of injuring the tongue papillae 46 can be reduced.

Further, when the longitudinal direction 43 of the rising portion 36′ is provided sideways in relation to the longitudinal direction 42 of the protrusion 41 to clean tongue coating by moving the head 1, the rising portion 36′ becomes sideways, so that the grime of the tongue coating can be surely rubbed out without failing to trap the grime of the tongue coating.

Furthermore, the side of the distal end 45 of the rising portion 36′ is folded toward the head 1 and thus when the main body of the cleaner is moved in the direction Z to clean the tongue coating, the rising portion 36′ becomes sideways and the distal end 45 is folded and therefore the tongue coating is enveloped with the protrusion 41, the rising portion 36′ and the distal end 45 folded, so that the grime can be more surely rubbed off.

Besides, the approximately central portion 36′A in the longitudinal direction 43 of the rising portion 36′ is formed into a circular-arc shape protruding upward and thus if the tongue papillae 46 is located at the end of the bridging portion 36′, when the linear material 41 is moving relatively to a tongue, the tongue papillae 46 can move relatively to the approximately central portion 36′A of the rising portion 36′. Hence, the rising portion 36′ is allowed to slip through the tongue papillae 46, so that the linear material 41 can slip through the tongue papillae 46 without injuring the tongue papillae 46.

### SIXTH EMBODIMENT

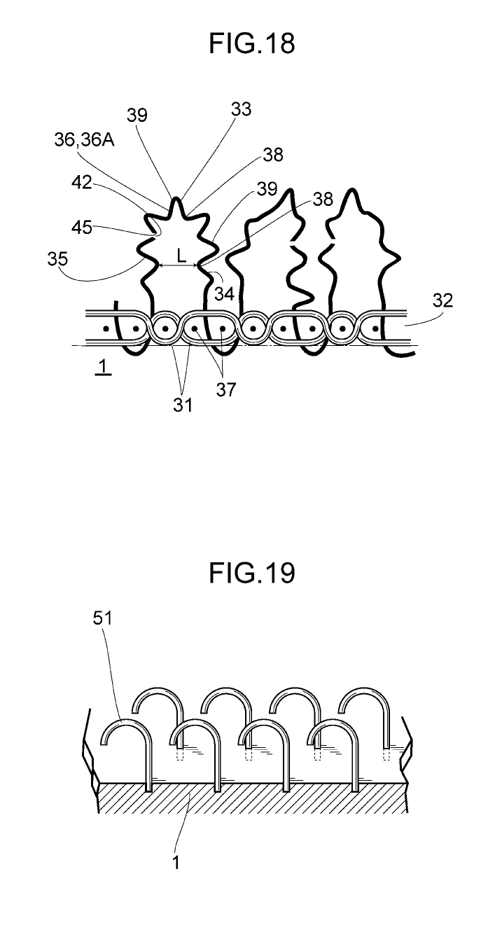

In a sixth embodiment shown in FIG. 18, the structures in the fifth and sixth embodiments are combined together. The disconnected portion 42 is formed in any one of the protrusions 34, 35 of the loop 33 provided continuously with depressed portions 38 and protruded portions 39 to make the distal end of a side of the bridging portion 36 forming the rising portion into a free end.

Accordingly, when the loop 33 is caught by the tongue papillae 46, the depressed portions 38 and the protruded portions 39 extend to act as a cushion and besides the distal ends 45 acting as a free end also deforms to the opposite side to the moving direction of the head 1, so that the tongue papillae 46 can be even more reliably prevented from being injured.

### SEVENTH EMBODIMENT

In a seventh embodiment shown in FIG. 19, inverted-J-shaped linear materials 51 are provided on the head 1 in a protruding manner. Note that the linear materials 51 may be formed from animal hairs such as those of a horse, a pig or the like in addition to synthesized resin.

Accordingly, when the linear materials catch the tongue papillae, a side of the distal end in each of the linear materials is pulled by the tongue papillae 46 to deform and thus the linear materials can slip through the tongue papillae, causing an effect of being capable of reducing the possibility of injuring the tongue papillae.

As described above, the tongue cleaner according to the present invention may be modified in various ways, such as by varying its shapes. For example, the head may have a flat surface instead of having the raised and valley portions.

### EIGHTH EMBODIMENT

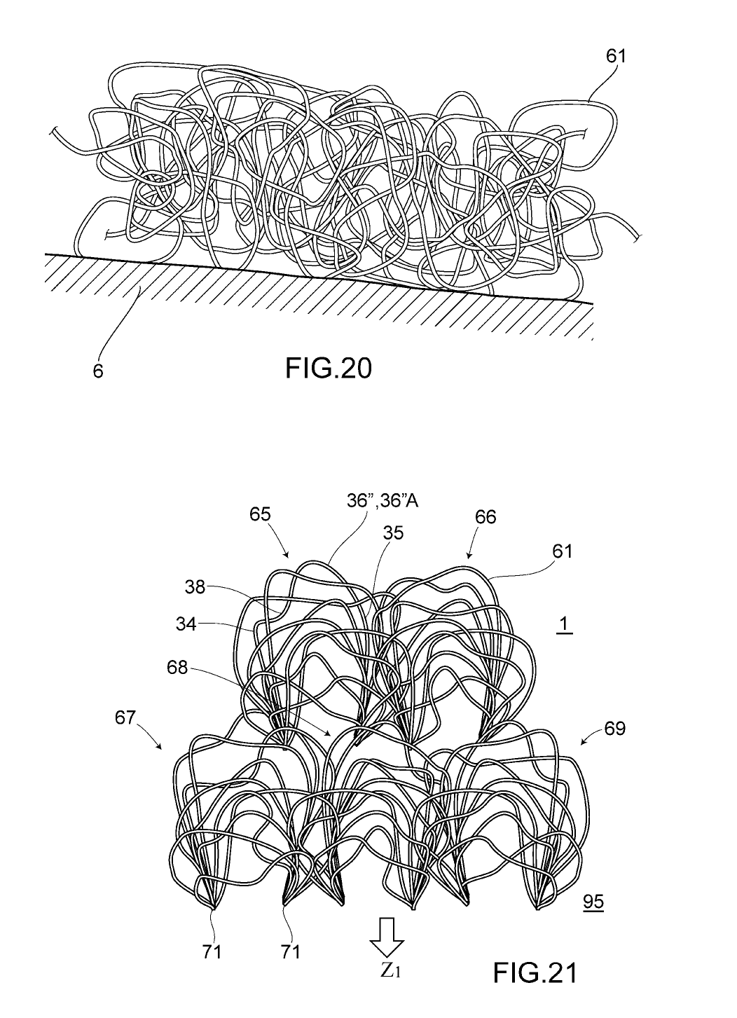

FIGS. 20 to 29 show an eighth embodiment using a loop-pile-woven textile as well as the fourth embodiment.

FIG. 20 shows linear materials 61 protruding from a main body of a tongue cleaner inside a ring 6. In the eighth embodiment, as shown in FIG. 20, the linear materials are randomly formed. The specific structure that the linear materials 61 are randomly formed, and the effects and the manufacturing method thereof are described hereunder with reference to FIGS. 21 to 41.

FIG. 21 shows a part of the linear materials 61 formed by a plurality of loop-piles protruding from the head 1 forming a main body of a cleaner. In FIG. 21, one linear material group is made up of a plurality of the linear materials 61 protruding from a pair of common loop-starting-points 71, 71. Here, FIG. 21 shows five linear material groups 65, 66, 67, 68, 69. In this example, ten linear materials 61 protrude from the pair of the common loop-starting-points 71, 71, while the number of the linear materials is not to be specifically limited, and there is no problem if only one linear material is provided. It is, however, preferable that a plurality of linear materials is provided. The linear material group 65 is located between the linear material groups 67 and 68 and posterior to them with respect to the moving direction Z₁. Likewise, the linear material group 66 is located between the linear material groups 68 and 69 and posterior to them with respect to the moving direction Z₁. Such arrangement of the linear material groups enables the posterior linear material groups to cover a gap left between the adjacent anterior linear material groups. Alternatively, the linear material groups may be aligned with respect to the moving direction Z₁, if the gap is covered by the deformation of the linear materials 61.

When focusing attention on one linear material group 67, it should be noted that the height and leaning direction of each linear material 61 are totally random. In addition, substantially all the loop shapes of the linear materials 61 are different from each other. The same can be said for the other linear material groups 65, 66, 68, 69.

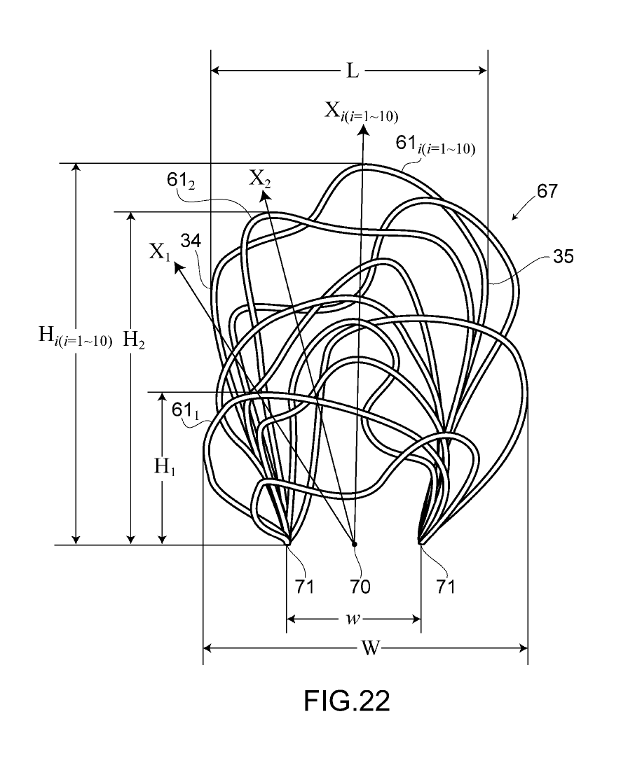

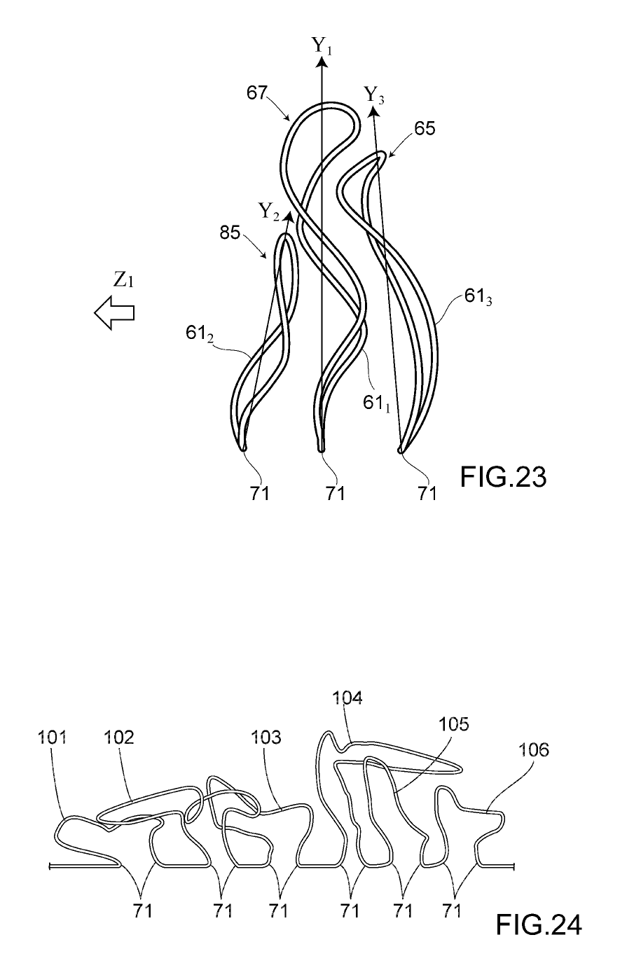

FIGS. 22 and 23 are enlarged views of the linear material group 67 shown in FIG. 21, providing definitions of the height and leaning directions of the linear material 61 described above. FIG. 22 is a view seen from a parallel direction with respect to the moving direction Z₁, and FIG. 23 is a view seen from the direction which intersects with the moving direction Z₁ (at a right angle in the present embodiment), or the direction of the interval L between the first and second protrusions 34, 35 in a plan view. The direction of the interval L is parallel with a width w defined as a distance between the pair of the common loop-starting points. Regarding each of the linear materials 61, Hᵢ represents the height thereof, Xᵢ represents the leaning direction thereof toward a horizontal direction as shown in FIG. 22, and Yₖ represents the leaning direction thereof toward a front-back direction as shown in FIG. 23.

In the example shown in FIG. 22, there are ten linear materials 61, and hence the symbol “i” represents an integer from 1 to 10. The height Hᵢ is defined as that of a certain position or point in each linear material 61 which is the highest from the loop-starting-point with respect to the moving direction Z₁. FIG. 22 shows H₁ and H₂ as examples. The direction Xᵢ is defined as the direction from a midpoint 70 between the two loop-starting-points 71, 71 toward the point of the height Hᵢ of each linear material 61. In FIG. 22, the directions X₁ and X₂ correspond to the heights H₁ and H₂, respectively.

Taking one linear material group 67 as an example, the heights Hᵢ of the linear materials 61 are different from each other. Due to such differences, a gap unable to be covered by each linear material 61 with respect to the moving direction Z₁ in an area occupied by the linear material group 67 can be minimized. The smaller the gap becomes, the more efficiently the grime of the tongue coating can be scraped out. Further, at least one of the comparatively high linear materials 61 in the linear material group 67 is allowed to overlap the region of the different linear material group 65, and thus there can be obtained the linear materials 61 which are more densely formed with respect to the moving direction Z₁. These configurations enable the grime of the tongue coating left without being scraped out to be reduced.

In each linear material group, the directions Xᵢ of the linear materials 61 are random. At least one linear material 61 protrudes toward the horizontal direction in FIG. 22. At least one linear material 61 protruded in that manner may overlap the different linear materials 61 of the adjacent linear material group 68. Because the linear materials 61 are randomly formed in the horizontal direction, a gap unable to be covered by each linear material 61 with respect to the horizontal direction can be minimized. The smaller the gap becomes, the more noticeably the grime of the tongue coating left without being scraped out can be reduced.

It is preferable that, in each linear material group, a width W defined as a distance, when measured in a side view of the FIG. 22, between the leftmost end and the rightmost end in all the linear materials 61 composing the linear material group is larger than a width w defined as a distance between the paired common loop-starting-points in each linear material group. This structure enables the grime of the tongue coating in wider areas to be rubbed off at once.

Next is a description of how the overlap in the direction of the height Hᵢ and the horizontal direction Xᵢ between the linear materials 61 occur. For example, in a case that one linear material 61 of the linear material group 67 intersects with one linear material 61 of the linear material group 68 in a plan view of FIG. 21, the two linear materials 61, 61 are referred to as overlapping each other. Even if the linear materials 61, 61 do not intersect with each other in a plan view, in a case that there is any crossover between the linear materials 61, 61 in a vertical direction in a space of the gap between the linear material groups 67, 68, then such two linear materials 61, 61 are still referred to as overlapping each other. The percentage of the linear materials 61 overlapping in the direction of the height Hᵢ and the horizontal direction Xᵢ with respect to the total linear materials 61 is preferably 60 to 100%, and more preferably 80 to 100%.

FIG. 23 shows the linear materials randomly formed in the leaning direction Yₖ (k represents an integer from 1 to 3 in FIG. 23) toward the front-back direction in one linear material group 67. Yₖ is defined as the direction from the loop-starting-point 71 toward the highest point of the linear material 61 in a side view of FIG. 23. At least one linear material 61 protrudes toward the moving direction Z₁, or toward the front-back direction. In FIG. 23, a direction Y₁ of one linear material 61₁ in the linear material group 67 is substantially vertical. In contrast, a direction Y₂ of the linear material 61₂ in the linear material group 85 located in front of the linear material group 67 leans toward the linear material group 67 (backward), and a part of the linear material 61₂ of the linear material group 85 overlaps the linear material 61₁ of the linear material group 67. In contrast, a direction Y₃ of the linear material 61₃ in the linear material group 65 located in the rear of the linear material group 67 leans toward the linear material group 67 (frontward), and a part of the linear material 61 of the linear material group 65 overlaps the linear material 61 of the linear material group 67. Accordingly, due to such random formation of the linear materials 61 in the front-back direction, a gap unable to be covered by each linear material 61 can be minimized with respect to the direction of the interval L between the first and second protrusions 34, 35. The smaller the gap becomes, the more noticeably the grime of the tongue coating left without being scraped out can be reduced.

Next is a description of how the overlap in the front-back direction Yₖ occurs. For example, in a case that one linear material 61 of the linear material group 67 intersects with one linear material 61 of the linear material group 85 in a side view of FIG. 23, the two linear materials 61, 61 are referred to as overlapping each other. Even if the linear materials 61, 61 do not intersect with each other in a plan view, in a case that there is any crossover between the linear materials 61, 61 in a vertical direction in a space of the gap between the linear material groups 67, 85, then the two linear materials 61, 61 are referred to as overlapping each other. The percentage of the linear materials 61 overlapping in the front-back leaning direction Yₖ with respect to the total linear materials 61 is preferably 40 to 100%, and more preferably 60 to 100%.

Referring to FIG. 24 showing one pile yarn from an anterior view, a linear material 102 overlaps two linear materials 101, 103, and the linear materials 101, 103 belong to the different linear material groups from that of the linear material 102 respectively. The linear material 101 is located at a left side of the linear material 102 and the linear material 103 is located at a right side of the linear material 103. In addition, a linear material 104 overlaps a next linear material 105, and further overlaps a linear material 106 next to the linear material 105. Thus, one linear material may overlap a plurality of the linear materials. Though FIG. 24 shows only the overlaps in the horizontal direction, the same can be said for the front-back direction.

As described above, each linear material 61 is formed in such a three-dimensional shape that its height Hᵢ and leaning direction Xᵢ toward the horizontal direction are random with respect to the moving direction Z₁, and the leaning direction Yₖ toward the front-back direction is random with respect to the direction of interval L. Accordingly, a gap unable to be covered by each linear material 61 with respect to the height direction, the horizontal direction, and the front-back direction can be minimized. This is extremely advantageous because the smaller the gap becomes, the more efficiently the grime of the tongue coating can be scraped out by using the whole loop-pile.

These height Hᵢ, leaning direction Xᵢ toward the horizontal direction, and leaning direction Yₖ toward the front-back direction differ from one loop to another, and each loop shape also differs from one another. However, there is not completely eliminated the possibility of the occurrence of the same heights Hᵢ, the same leaning direction Xᵢ toward horizontal directions, or the same leaning direction Yₖ toward front-back directions, nor the possibility that the loop shape is totally or partially the same.

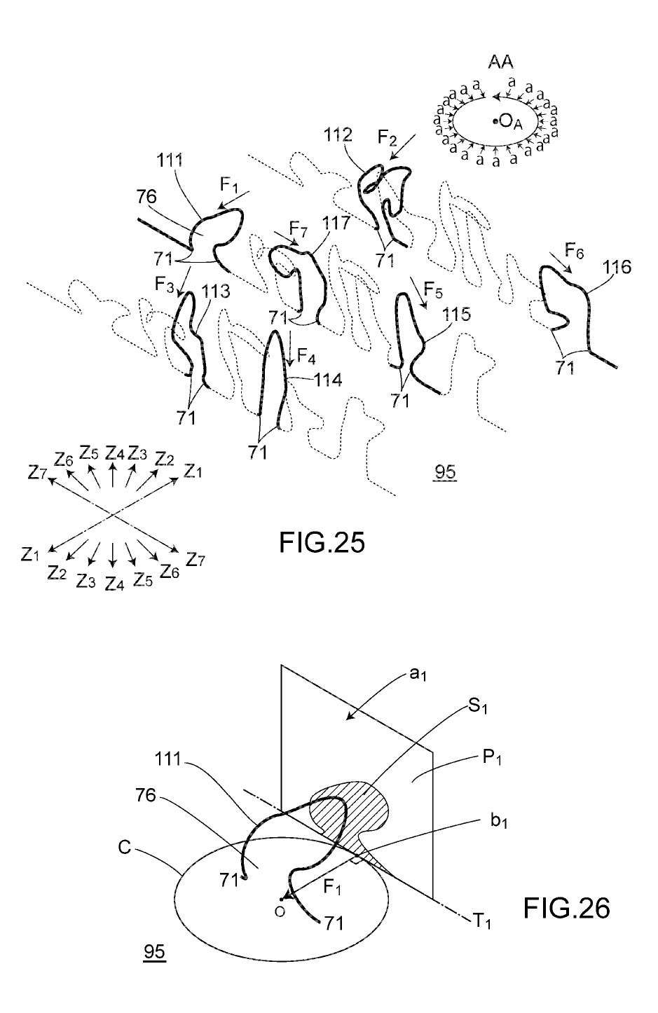

FIG. 25 shows the linear materials more randomly formed. Although all the loops 76 formed by the linear materials are illustrated as facing toward substantially the same moving direction Z₁ (the orientation F₁ in FIG. 25) in FIG. 22, the loops 76 formed by the linear materials 111 to 117 face toward mutually different directions F₁ to F₇, respectively, in FIG. 25 in which, for the purpose of illustration, an interval between the linear materials in the front-back direction is more widely illustrated than in reality, and some linear materials in the horizontal direction are omitted.

In fact, some linear materials have a plurality of loops therein each of which faces toward a direction different from the others'. Here, for the purpose of illustration, the orientation Fᵢ of the loop 76 is assumed as the direction in which a maximum amount of grime of the tongue coating can be rubbed off when moved to such direction. Specifically, when the linear material is seen from directions a, or directions from certain points on all around the arrow AA toward a center point O_{A}, and the arrow AA has a precise circle shape formed on a plane parallel to the sheet 95 in FIG. 25, then the orientation F of the loop 76 is defined as the direction from a contact b on the circumference of circle of the arrow AA to the center point O_{A} of the precise circle, and the contact b is a point where a projected area of a region surrounded by the linear material becomes the largest when the linear material is projected onto a plane P including a tangent line T and perpendicular to the sheet 95 in each direction a. This definition is described in more detail with reference to FIGS. 26 to 28.

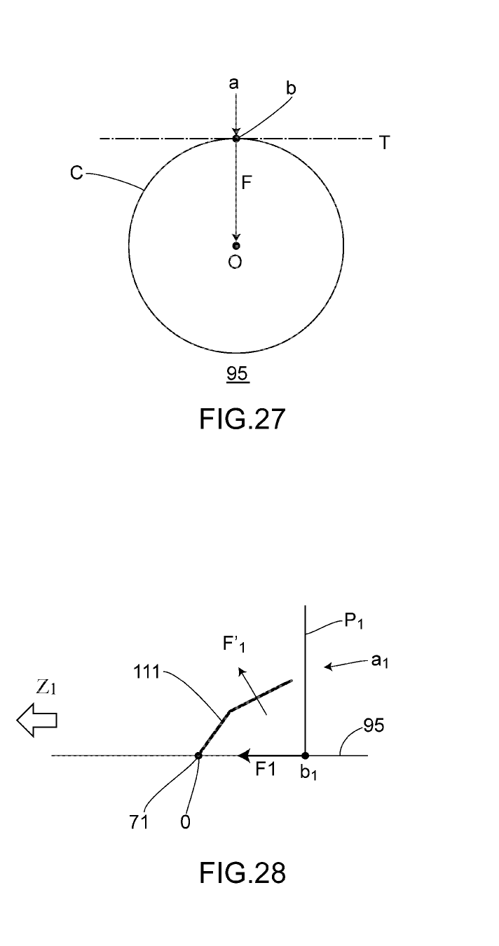

FIG. 26 illustrates a process of giving the definition of the orientation F₁ of the loop 76 formed by the linear material 111. At first, there is formed a circle C whose center is a center point O which is a middle point between the loop-starting points 71, 71 on a plane parallel to the sheet 95. FIG. 26 shows a case that the direction a is a direction a₁. A plane P₁ is a plane including a tangent line T₁ of the circle C at direction a₁ and perpendicular to the sheet 95. A projected area S₁ is an area when the loop 76 of the linear material 111 is projected onto the plane P₁. The orientation F₁ of the loop 76 of the linear material 111 is defined as the direction from a contact b₁ of the circle C and the tangent line T₁ to the center point O of the circle C when the direction in which the projected area becomes the largest is the direction a₁ as a result of a comparison among the projected areas of loop 76 formed by the linear material 111 at all directions a on the circumference of the circle C. In reality, however, it is sufficient if the directions within a range of 0 to 180 degrees are considered instead of all directions (0 to 360 degrees). FIG. 27 is an overhead view with respect to the sheet 95, and shows a relation between the circle C, and the tangent line T and the orientation F of the loop when the direction a is selected.

Further, FIG. 28 is a lateral view of the linear material 111. The linear material leans to a direction along a moving direction Z₁, and in fact the loop 76 is observed as being directed toward an obliquely upward direction F′₁. However, the orientation F₁ is included in a plane parallel to the sheet 95 since the orientation F₁ is the direction from the contact b of the circle C on the plane parallel to the sheet 95 toward the center point O.

According to the above definition, in the case of the linear material 111, when the tongue cleaner is moved to the moving direction Z₁ identical with the orientation F₁, the maximum amount of grime of the tongue coating can be rubbed off.

In FIG. 25, the direction defined for descriptive purposes is the same direction as the moving direction Z₁, and the orientation F₂ is a 15-degree angle, the orientation F₃ is a 30-degree angle, the orientation F₄ is a 45-degree angle, the orientation F₅ is a 60-degree angle, the orientation F₆ is a 75-degree angle, and the orientation F₇ is a 90-degree angle with respect to the orientation F₁. This structure enables the grime of the tongue coating to be surely rubbed off by the loop 76 facing to the moving direction even if the tongue cleaner is moved to any moving directions Z₁ to Z₇. Here, the angles of the directions F of the loops 76 differ every 15 degrees, but the linear materials are deformed so that the directions of the loops 76 face in the directions covering all angles.

In FIG. 25, the same structure as that of between the moving directions Z₁ and Z₇ (from 0 to 90-degree angles with respect to the moving direction Z₁) can be applied to that of between the moving directions Z₇ and Z₁ (from 90 to 180-degree angles with respect to the moving direction Z₁), and thus the linear materials are deformed so that the directions of the loops 76 face in the directions covering all directions denoted by the arrow AA.

The directions of the loops 76 may face in the different directions in one linear material group, while each of the linear materials 111, 112, 113, 114, 115, 116, 117 belongs to the different linear material groups from one another. Further, the directions of the loops may face in the different directions in one linear material. That is to say, the orientation F of the loop 76 according to the present invention includes the directions of all loops enable to rub off the grime of the tongue coating. It is important to the completed product of the tongue cleaner that the directions of the loops cover substantially all directions. This structure enables the grime of the tongue coating to be surely rubbed off even if the tongue cleaner is moved to any directions.

In the present embodiment, substantially the same manufacturing method as the fourth embodiment can be employed. That is, the rotating brush 90 is pressed against the linear materials 61. This process may be provided after setting the sheet 4, 5 on the surface of the head 1. However, it is preferable that the rotating brush fixed in place is pressed against the looped linear materials 61 (textile 13) in a state of texture (sheet 95) prior to being mounted on the head 1 as shown in FIG. 4, thereby deforming the linear materials 61.

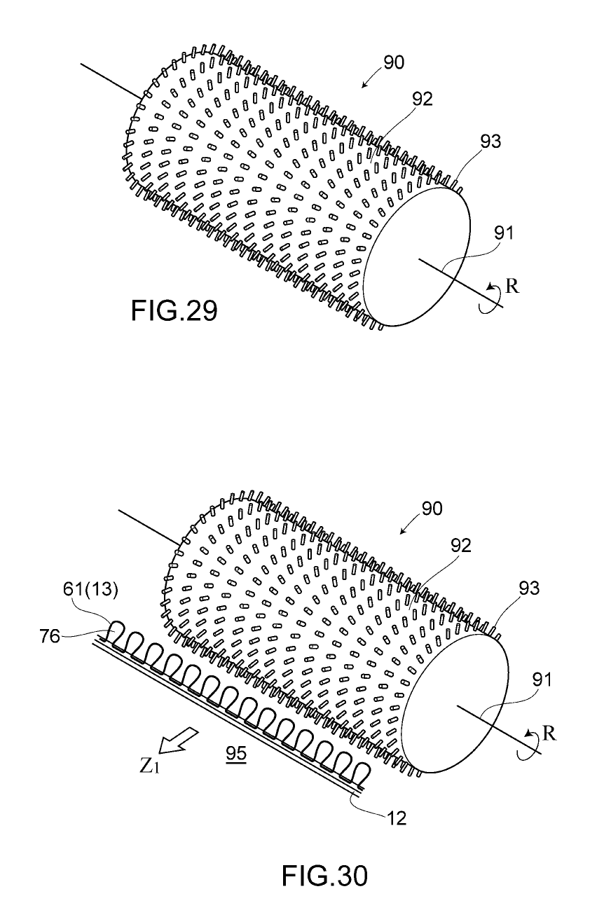

FIG. 29 shows an example of the rotating brush 90. The rotating brush comprises a rotating drum 92 which can rotate in a rotational direction R with a rotating shaft 91, and a plurality of pins 93 arranged on the rotating drum 92. In the present embodiment, a plurality of pins 93 is arranged evenly spaced apart along a circumferential direction of the rotating drum 92 and an axial direction of the rotating shaft 91. However, pins 93 may be randomly arranged.

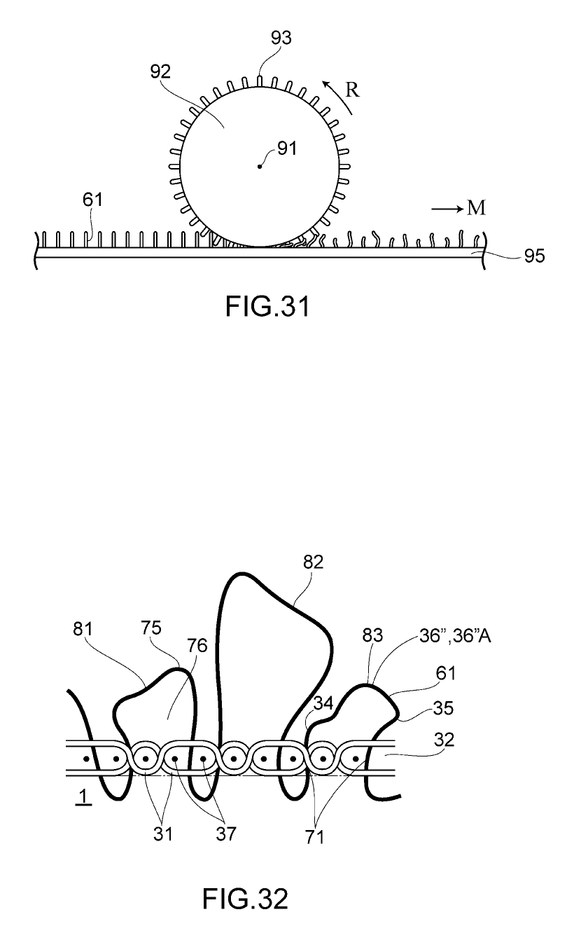

FIG. 30 is a perspective view illustrating the rotating brush 90 being pressed against the sheet 95 with the direction of the interval L of the sheet 95 parallel to the axial direction of the rotating shaft 91 of the rotating brush 90, and FIG. 31 is a lateral view illustrating the same process as FIG. 30. The sheet 95 travels toward a direction M with being expanded to the plane state and being pressed by the rotating brush 90 on the lower side thereof. It is preferable that a speed at which the sheet 95 travels toward the direction M is controlled to be slower than a rotating speed at which the rotating brush 90 rotates in a rotational direction R. This control enables the loops 76 of the linear materials 61 of the sheet 95 to be readily caught by the pins 93 of the rotating brush 90. It should be noted that when the rotating brush 90 is pressed against the sheet 95, the rotating brush 90 may travel with rotating in one direction in a state of the sheet 95 being fixed in place.

Firstly, the loop 76 of the linear material 61 of the sheet 95 is caught by the pin 93 of the rotating brush 90, followed by extension of the linear material 61 toward the rotational direction R of the rotating brush 90, the linear material 61 being constructed of plastics material such as nylon. Next, after the linear material 61 is released from the pin 93, the linear material 61 would go back to the original shape and then shrinks. Some shrunk linear materials 61 fall to the side of the direction to which the linear materials 61 are extended, while some shrunk linear materials 61 go back to substantially the same position as the original one. Further, in some cases, the linear materials 61 may fall to the side opposite to the direction to which the linear materials 61 are extended with its rebound. Thus, a depressed portion 38 and a protruded portion 39 are formed in the shrunk linear materials 61. In addition, because there is a difference, with respect to each linear material 61, in how the loop 76 of the linear material 61 is caught by the pin 93 of the rotating brush 90, when the loop 78 is released from the pin 93, how much pressure the linear material 61 receives from the rotating brush 90, or the like, the height Hᵢ, leaning direction Xᵢ toward the horizontal direction, leaning direction Yₖ toward the front-back direction, and orientation F of the loop 76 of each linear material 61 become random after processing the sheet 95 by using the rotating brush 90. It is effective to control, for example, a configuration, size, rotating speed, and pressure pressed against the sheet 95, of the rotating brush 90.

The sheet 95 may travel under the rotating brush 90 more than once. In addition, the sheet 95 may travel under the rotating brush 90 after being turned, for example, 180 degrees or 90 degrees. Such number of times and direction that the sheet 95 travels can be combined freely. It is preferable that the rotating brush 90 is pressed from multiple directions with respect to the sheet 95 when the rotating brush 90 is pressed against the positionally-fixed sheet 95. These adjustments enable each linear material 61 to be formed in a three-dimensional shape well, and further the height Hᵢ, leaning direction Xᵢ toward the horizontal direction, leaning direction Yₖ toward the front-back direction, and orientation F of the loop 76 of each linear material 61 can become more random.

As shown in FIG. 32, one linear material 81 of the linear material group 67, one linear material 82 of the linear material group 68, and one linear material 83 of the linear material group 69 are comprised of one pile yarn. Accordingly, for example, when the linear material 82 is pulled hard toward the rotational direction R of the rotating brush 90, the adjacent linear materials 81, 83 may be pulled by the linear material 82. Such case is also one of the factors that the height Hᵢ, the leaning direction Xᵢ toward the horizontal direction, and the leaning direction Yₖ toward the front-back direction becomes random.

As described above, the depressed portion 38 and the protruded portion 39 of linear material 61 are formed as a result of the shrink of the linear material 61 after once it is extended. Therefore, the depressed portion 38 and the protruded portion 39 are formed to have not sharp angle but curved shape, and hence loop pile comprises curved part 75. Thus, each loop 76 can readily slip through the tongue papillae 40 without injuring the tongue papillae 40.

As above stated, according to the eighth embodiment of the tongue cleaner, the height Hᵢ, leaning direction Xᵢ toward the horizontal direction, leaning direction Yₖ toward the front-back direction, and orientation F of the loop 76 become random after processing the linear materials 61 by the rotating brush 90, thereby enabling the gap to be smaller. Accordingly, a wider area can be cleaned without remaining spaces which are not cleaned, and thus the more amount of grime of the tongue coating can be scraped out efficiently.

Further, in some linear materials 61, the approximately central portion 36″A in the longitudinal direction of the bridging portion 36″ is formed into a circular-arc shape protruding upward. Hence, if the tongue papillae 40 are located at the end of the bridging portion 36″, the tongue papillae 40 can move relatively toward the approximately central portion 36″A of the bridging portion 36″ during the movement of the loop 76 toward to the tongue, and therefore the length over which the loop 76 can slip through the tongue papillae 40 can be enlarged, so that the loop 76 can slip through the tongue papillae 40 without injuring the tongue papillae 40.

In addition, the shape and the percentage of the linear materials 61 can be applied to not only the case of the sheet 95 after processed by the rotating brush 90 but also the case of the completed product of the tongue cleaner. Further, if the linear material can be shrunk after extending the linear material 61 by catch the loop 76, the method to do so is not limited to the one using the rotating brush 90. For example, there can be employed a method in which the surface of the sheet 95 is bushed by a scrub brush, a wire-brush or the like.

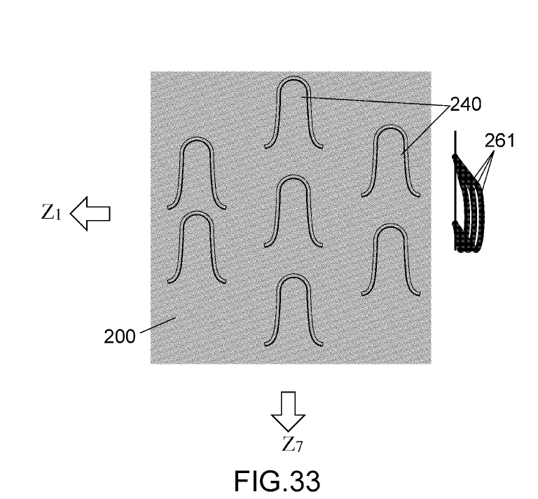

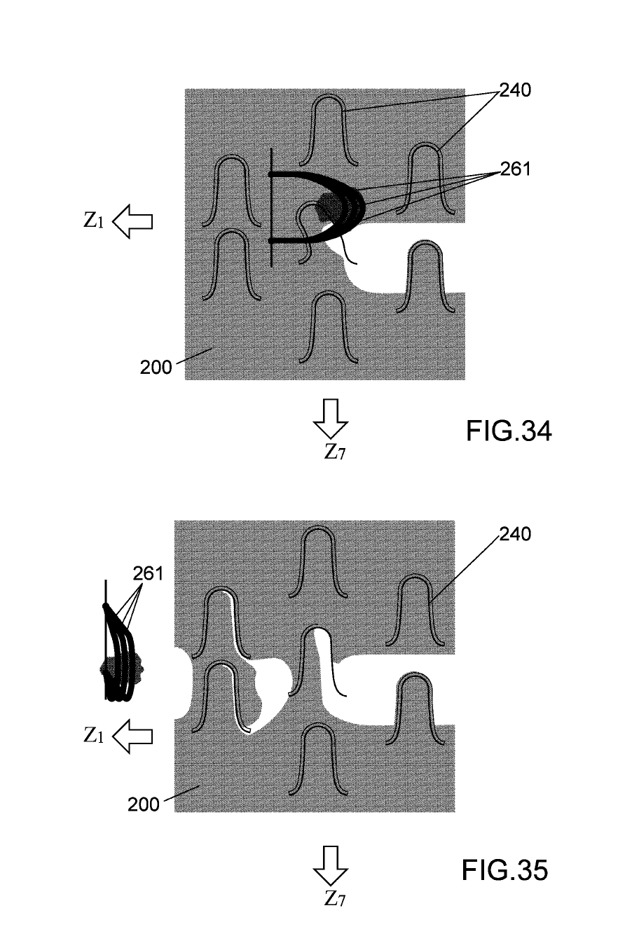

FIGS. 33 to 35 show how the grime 200 of the tongue coating is rubbed off by the tongue cleaner of the conventional art. As shown in FIG. 33, the grime 200 of the tongue coating is stuck to the entire surface of the tongue and the entire surface of the papillae 240. Here, the linear materials 261 of the conventional art are of substantially same shapes. In FIG. 34, the tongue cleaner is moved with being pressed against the surface of the tongue, and the grime 200 of the tongue coating is rubbed off by the linear materials 261. However, because the linear materials 261 can only move into fewer gaps between papillae and remove fewer grime. As a result, as shown in FIG. 35, the grime 200 of the tongue coating which was not rubbed off remains even after the linear materials 261 had passed.

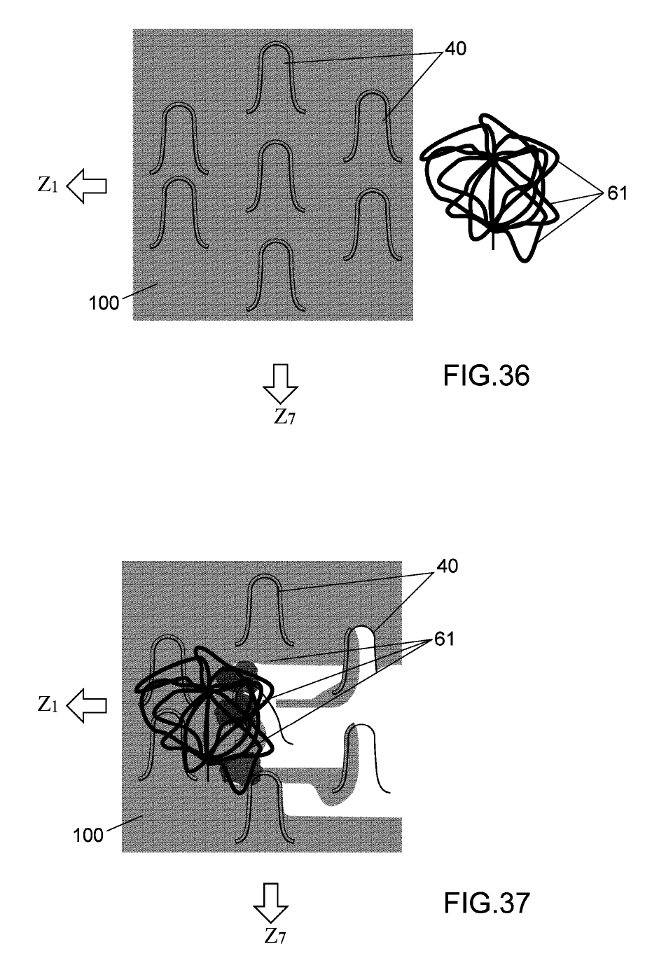

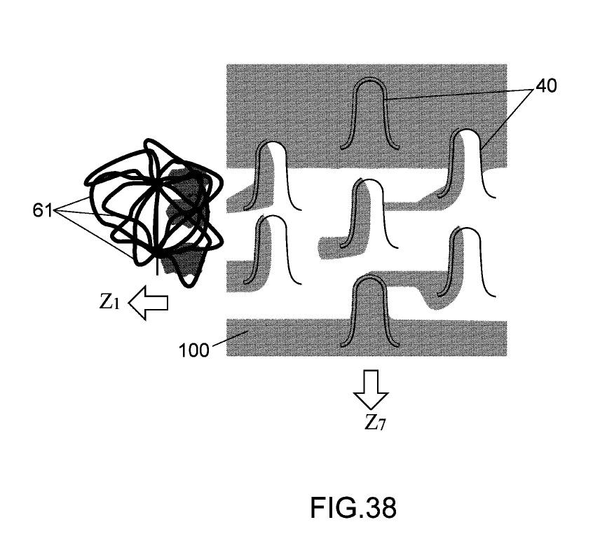

In contrast, FIGS. 36 to 38 show how the grime 100 of the tongue coating is rubbed off by the tongue cleaner according to the present invention. In FIG. 36, the grime 100 of the tongue coating is stuck to the entire surface of the tongue and the entire surface of the papillae 40 like FIG. 33 illustrating the conventional art. The linear materials 61 are formed into different shapes, and randomly formed in the height direction, the horizontal direction, and the front-back direction. Accordingly, as shown in FIG. 37, when the tongue cleaner is moved with being pressed against the surface of the tongue, the linear materials 61 can move into small gaps between the papillae 40. As a result, as shown in FIG. 38, few grime 100 of the tongue coating remain after the linear materials 61 had passed, thereby enabling the grime 100 of the tongue coating to be more surely scraped out.

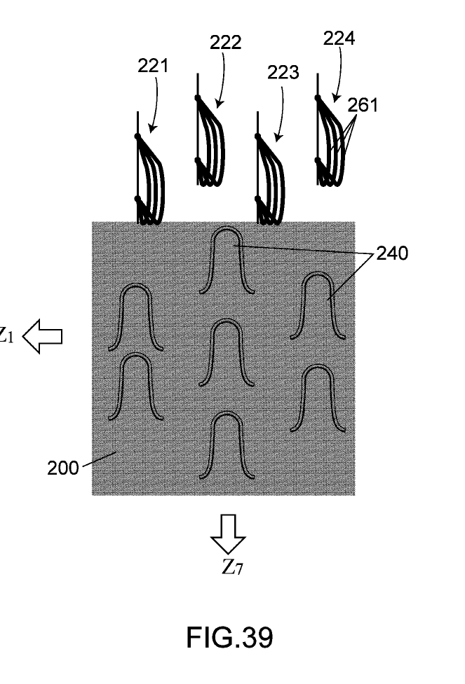

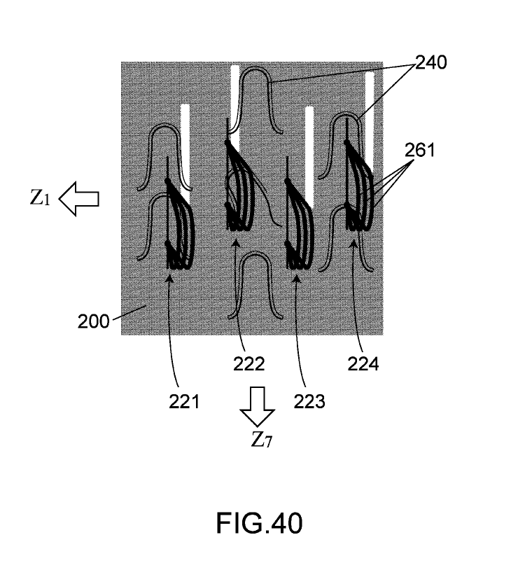

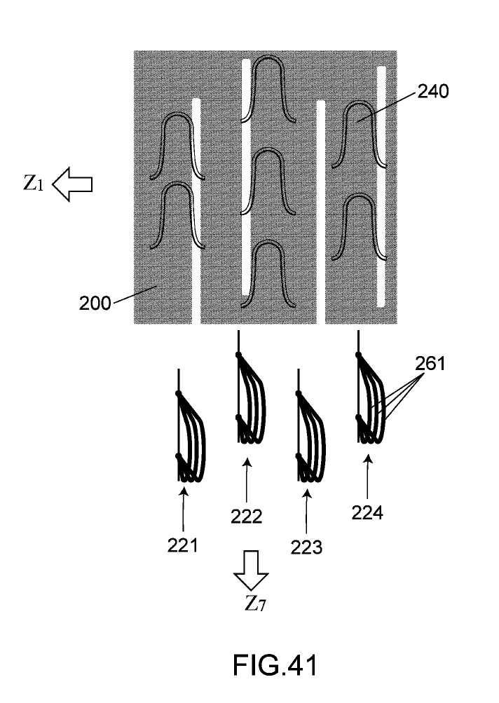

FIGS. 39 to 41 show how the grime 200 of the tongue coating is rubbed off by moving the tongue cleaner of the conventional art toward the moving direction Z₇ perpendicular to the moving direction Z₁ with the tongue cleaner being faced to the same direction as the one shown in FIGS. 33 to 35. As shown in FIG. 39, the face of the loop formed by the linear materials 261 of the linear material groups 221, 222, 223, 224 is all directed to the moving direction Z₁. Accordingly, as shown in FIG. 40, even if the tongue cleaner is moved to the moving direction Z₇ with being pressed against the surface of the tongue, the linear materials 261 can hardly remove grime 200 of the tongue coating. As a result, as shown in FIG. 41, the grime 200 of the tongue coating which was not rubbed off remains even after the linear materials 261 had passed.

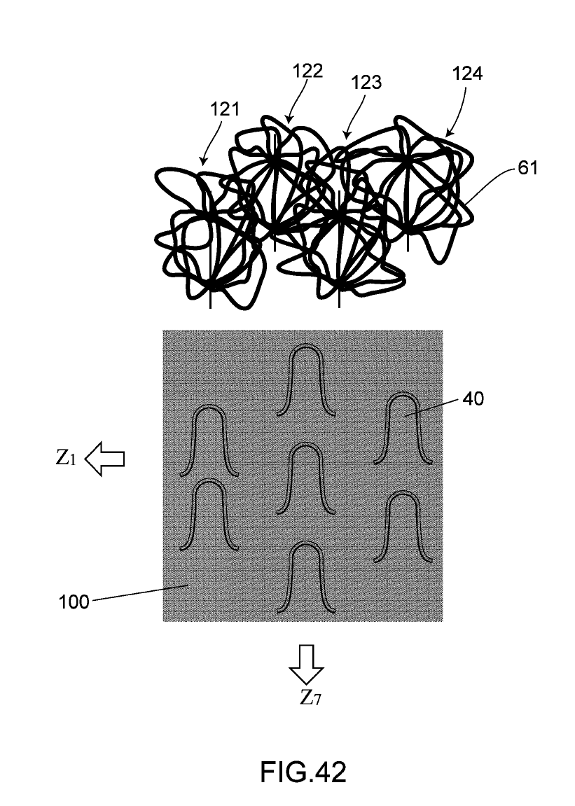

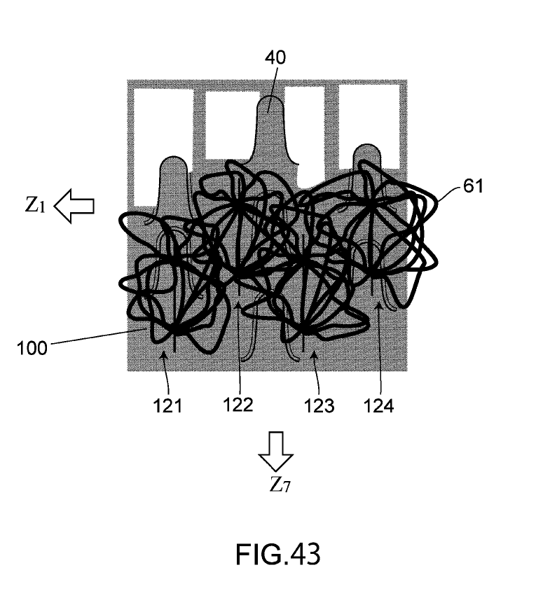

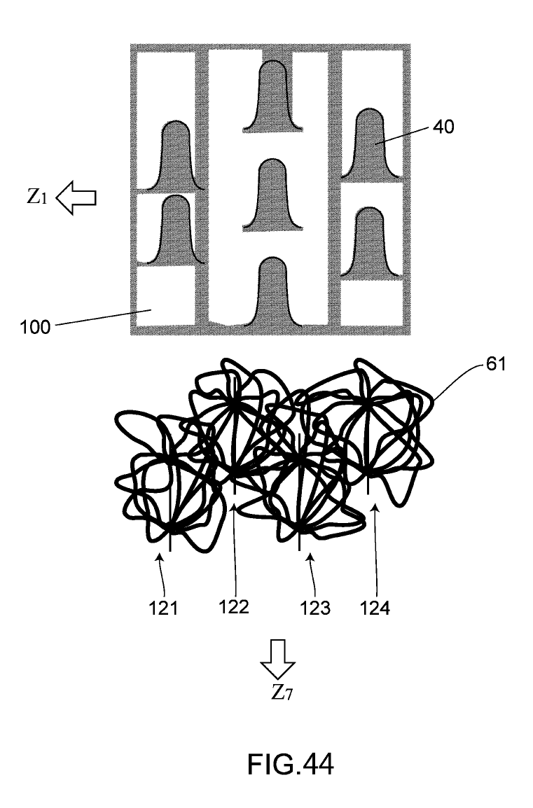

In contrast, FIGS. 42 to 44 show how the grime 100 of the tongue coating is rubbed off by moving the tongue cleaner according to the present invention toward the moving direction Z₇ perpendicular to the moving direction Z₁ with the tongue cleaner being faced to the same direction as the one shown in FIGS. 36 to 38. As shown in FIG. 42, the loops formed by the linear materials 61 of the linear material groups 121, 122, 123, 124 face in the various directions. The linear materials are formed into the different shapes from one another, and the height, the leaning direction toward the horizontal direction, the leaning direction toward the front-back direction thereof, and the orientation of the loop are random. Accordingly, as shown in FIG. 43, if the tongue cleaner is moved to the moving direction Z₇ while being pressed against the surface of the tongue, the linear materials 61 can rub off the grime 10 of the tongue coating with respect to the moving direction Z₇ as well as the moving direction Z₁. As a result, as shown in FIG. 44, few grime 100 of the tongue coating remain after the linear materials 61 passed, thereby enabling the grime 100 of the tongue coating to be more surely scraped out.

We claim:

1. A tongue cleaner comprising:
a head forming a main body of said tongue cleaner; and
a plurality of linear materials protruding from said head and comprised of a loop pile;
wherein said plurality of the linear materials make up a plurality of linear material groups comprised of at least two of said linear materials protruding from a pair of common loop-starting points, and
a loop shape of said linear material differs from one another in each linear material group.

2. The tongue cleaner according to claim 1, wherein each linear material in the plurality of the linear material groups has a random height, random leaning directions toward both horizontal and front-back directions as well as a random orientation of the loop formed by said linear material.

3. The tongue cleaner according to claim 2, wherein each linear material in the plurality of the linear materials has a random height, random leaning directions toward both horizontal and front-back directions as well as a random orientation of the loop formed by said linear material.

4. The tongue cleaner according to claim 2, wherein the orientation of each loop is so random as to cover substantially all directions in each linear material group.

5. The tongue cleaner according to claim 3, wherein the orientation of each loop is so random as to cover substantially all directions in the linear materials.

6. The tongue cleaner according to claim 2, wherein the plurality of the linear material groups include first and second linear material groups, and at least one linear material of the first linear material group overlaps at least one linear material of the second linear material group with respect to a moving direction (Z₁) of said tongue cleaner.

7. The tongue cleaner according to claim 3, wherein the plurality of the linear material groups include first and second linear material groups, and at least one linear material of the first linear material group overlaps at least one linear material of the second linear material group with respect to a moving direction (Z₁) of said tongue cleaner.

8. The tongue cleaner according to claim 2, wherein the plurality of the linear material groups include first and third linear material groups, and at least one linear material of the first linear material group overlaps at least one linear material of the third linear material group with respect to a direction parallel with a width defined as a distance between the pair of the common loop-starting points.

9. The tongue cleaner according to claim 3, wherein the plurality of the linear material groups include first and third linear material groups, and at least one linear material of the first linear material group overlaps at least one linear material of the third linear material group with respect to a direction parallel with a width defined as a distance between the pair of the common loop-starting points.

10. The tongue cleaner according to claim 2, wherein the plurality of the linear material groups include at least one linear material group in which a width (W) defined as a distance between a leftmost end and a rightmost end in said one linear material group is larger than a width (w) defined as a distance between the pair of the common loop-starting-points therein.

11. The tongue cleaner according to claim 3, wherein the plurality of the linear material groups include at least one linear material group in which a width (W) defined as a distance between a leftmost end and a rightmost end in said one linear material group is larger than a width (w) defined as a distance between the pair of the common loop-starting-points therein.
